(19) United States
(12) Patent Application Publication
(10) Pub. No.: US 2005/0142634 A1
(43) Pub. Date: Jun. 30, 2005

(54) Novel modulator of non-genomic activity of nuclear receptors (mnar) and uses thereof

(75) Inventors: Boris Joseph Cheskis, North Wales, PA (US); Elliott Bruce Nickbarg, Belmont, MA (US); Chi-Wai Wong, Hayward, CA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 10/473,753

(22) PCT Filed: Apr. 3, 2002
(86) PCT No.: PCT/US02/10624

Related U.S. Application Data
(60) Provisional application No. 60/281,155, filed on Apr. 3, 2001.

(51) Int. Cl. C12Q 1/68; A01K 67/027; C07H 21/04; C12N 1/18; C07K 14/72; C07K 16/28

(52) U.S. Cl. 435/69.1; 435/320.1; 435/325; 435/254.2; 800/14; 530/358; 536/23.5; 435/6; 530/388.22

(57) ABSTRACT

A novel protein modulator of non-genomic activity of nuclear receptors (MNAR), which interacts with estrogen receptors alpha (ERα), estrogen receptor beta (ERβ), and other nuclear receptors, was discovered and characterized. An MNAR-ER complex, in vitro and in vivo, relates to nucleic acids, antisense, recombinant expression of nucleic acids, as well as host cells, compositions and assays that utilize the nucleic acids and protein of this invention.

This application claims the benefit of U.S. application Ser. No. 60/281,155, filed Apr. 3, 2001. This application is a continuation in part of the aforementioned patent application, the content of which is incorporated by reference.

### FIELD OF THE INVENTION

The present invention relates to the fields of biochemistry and internal medicine and to a novel modulator of non-genomic activity of nuclear receptors (MNAR), which interacts with both estrogen alpha (ERα) and estrogen beta (ERβ). More particularly, this invention concerns novel compositions, methods, and assays for modulating the transcriptional activity of estrogen receptors and/or the activation of the src family of tyrosine kinases. This invention also relates to novel compositions, methods, and assays for differentiating between genomic and non-genomic activity of nuclear receptors (e.g. estrogen receptors). Additionally, this invention relates to nucleic acids encoding MNAR, antisense, recombinant expression of nucleic acids, as well as host cells, compositions, and assays that utilize the nucleic acids and protein of this invention.

### BACKGROUND OF THE INVENTION

Nuclear hormone receptors are a superfamily of ligand-inducible transcription factors, which, as a class, are involved in ligand-dependent transcriptional control of gene expression. Binding of a specific ligand, inducing conformational changes in the receptor molecule, affects receptor interaction with other transcription factors, and, ultimately, formation of the preinitiation complex. This process regulates the rate of gene transcription. (D. J. Mangelsdorf, et al., Cell 83: 835-9, 1995).

The nuclear hormone receptor superfamily includes steroid hormone receptors, non-steroid hormone receptors, and orphan receptors. The receptors for glucocorticoids (GR), mineralcorticoids (MR), progestins (PR), androgens (AR), and estrogens (ER) are examples of classical steroid receptors. In addition to steroid hormone receptors, this nuclear hormone receptor superfamily consists of receptors for non-steroid hormones, such as vitamin D, thyroid hormones, and retinoids. Moreover, a range of nuclear receptor-like sequences have been identified which encode so called “orphan” receptors. These orphan receptors are structurally related to and, therefore, classified as nuclear hormone receptors, although no putative ligands have been identified yet. (Cell, 83: 851-857, 1995).

The superfamily of nuclear hormone receptors is characterized structurally and functionally by a modular structure in comprising six distinct structural and functional domains, A to F. More specifically these receptors have a variable N-terminal region (domain A/B); followed by a centrally located, highly conserved DNA-binding domain (hereinafter referred to as DBD; domain C); a variable hinge region (domain D); a conserved ligand-binding domain (hereinafter referred to as LBD; domain E); and a variable C-terminal region (domain F). Cell, supra.

The N-terminal region, which is highly variable in size and sequence, is poorly conserved among the different members of the superfamily. This part of the receptor is involved in the modulation of transcription activation.

The DBD consists of approximately 66 to 70 amino acids and is responsible for DNA-binding activity: This domain targets the receptor to specific DNA sequences, called hormone responsive elements (hereinafter referred to as HRE), within the transcription control unit of specific target genes on the chromatin. Steroid receptors such as GR, MR, PR, and AR recognize similar HRE DNA sequences, while the ER recognizes a different DNA HRE sequence. After binding to DNA, the steroid receptor is thought to interact with components of the basal transcriptional machinery and with sequence-specific transcription factors, thereby modulating the expression of specific target genes.

The LBD is located in the C-terminal part of the receptor and is primarily responsible for ligand binding activity. In this way, the LBD is essential for recognition and binding of the hormone ligand and, in addition, possesses a transcription activation function, thereby determining the specificity and selectivity of the hormone response of the receptor. Although moderately conserved in structure, the LBD's are known to vary considerably in homology among the individual members of the nuclear hormone receptor superfamily.

When a hormone ligand for a nuclear receptor enters the cell and is recognized by the LBD, it will bind to the specific receptor protein, thereby initiating an allosteric alteration of the receptor protein. (Cell, supra). As a result of this alteration the ligand/receptor complex switches to a transcriptionally active state and, as such, is able to bind through the presence of the DBD with high affinity to the corresponding HRE on the chromatin DNA. In this way, the ligand/receptor complex modulates expression of the specific target genes. The diversity achieved by this family of receptors results from their ability to respond to different ligands.

In addition to modulation of genomic activity, hormone receptor complexes can have important and varied non-genomic effects. This non-genomic activity is characterized by fast and transient increase in intracellular second messengers. In turn, these second messengers are involved in a number of different pathways and cascades that affect cellular functions, such as proliferation and differentiation.

More generally, steroid hormone receptors are connected with embryonic development, adult homeostasis, and organ physiology. Various diseases and abnormalities are ascribed to a disturbance in steroid hormone action. Since steroid receptors exercise their influence as hormone-activated transcription modulators and as hormone-activated stimulators of non-genomic activity, further investigation into various approaches to modifying, interacting with, or modulating these receptors is an area of immense significance. For example, mutations and defects in these receptors, as well as overstimulation or blocking of these receptors, may provide better insight into the underlying mechanism of the hormone signal transduction pathway, thereby leading to increased efficacy in treatment of a wide variety of steroid receptor-linked diseases and abnormalities.

Estrogen Receptors

Estrogen (E2) exerts numerous biological effects in different tissues through an interaction with the ER. Amino acid sequence analyses, transient transfection studies, and mutational dissections of ER indicate that ER has the classical modular structure described above. The N-terminal A/B domain of the ER contains a transactivation function, referred to as transcriptional activation function 1 (TAF-1). The DBD contains two zinc fingers and is responsible for DNA recognition. The LBD and a second transactivation function, referred to as TAF-2, is located at the C-terminal of ER.

Upon binding to hormone, the ER undergoes an activation and transformation step. The activated ER interacts with specific estrogen response elements (EREs) that are located in the promoter region of estrogen-regulated genes and that influence its target gene transcription. Over the past decade, numerous studies have provided a basic understanding of both the effects of ligand (agonist/antagonist) on the ER and the relationship between the structure and function of the ER. Nevertheless, little is known regarding the mechanisms for the non-genomic activity of ER.

ERβ appears to be distinct from the more commonly known estrogen receptor, referred to as ERα. Collectively, ERα and ERβ are referred to herein as ER. The DBD of ERβ is 90% identical to that of ERα. However, the overall homology between the ligand binding domain (LBD) of ERα and ERβ is less than 55%. Like ERα, ERβ can stimulate transcription from an ERE in a ligand-dependent manner.

It has been established that estrogens induce fast and transient increases in the levels of intracellular second messengers, including calcium and cAMP, and that estrogens induce activation of mitogen-activated protein kinase (MAPK) and phospholipase C (Collins and Webb, 1999). In fact, numerous studies have demonstrated that estrogens induce rapid and transient activation of the Src/Ras/MAP kinase phosphorylation pathway. Activation of this pathway triggers vital cellular functions including cell proliferation and differentiation. The time course of these acute events parallels that elicited by peptide hormones, thus supporting the hypothesis that these events do not involve the ‘classical’ genomic action of estrogens.

Recent data also suggests a direct link between the estrogen receptor and the mitogen-activated protein (MAP) kinase-signaling cascade. MAP kinases are a family of serine-threonine kinases that are phosphorylated and activated in response to a variety of signals. These enzymes transduce extracellular signals from multiple membrane receptors to intracellular targets, including transcription factors, cytoskeletal proteins and enzymes. The MAP kinase family includes the extracellular-signal related kinases (ERKs), p38 and cJun N-terminal kinases which signal through a pathway involving sequential activation of Ras, Raf and mitogen-activated protein kinase (MEK). (S. M. Thomas, J. S. Brugge, Annual Review of Cell & Developmental Biology 13, 513-609, 1997). In pulmonary endothelial cells, neuronal cells, osteoblasts and osteoclasts, 17β-estradiol (E2) has been reported to rapidly activate the MAPK pathway.

More specifically, in pulmonary endothelial cells, 17β-estradiol (E2) has been reported to rapidly stimulate nitric oxide (NO) production, thus explaining the ability of E2 to induce acute dilation of blood vessels. Chen et al. (Chen et al., 1999) have recently reported that E2 induces rapid activation of endothelial nitric oxide synthase (eNOS) in isolated pulmonary endothelial cells. Estrogen activation of eNOS was shown to occur through the rapid activation of the MAP kinase pathway. Complimentary studies have also shown that E2 induces calcium-dependent translocation of eNOS from the plasma membrane to intracellular sites close to the nucleus—an action that is rapid (within 5 minutes), receptor-mediated but nongenomic (Goetz et al., 1999). In addition, ligand-dependent ERα interaction with the p85α regulatory subunit of phosphatidylinositol-3-OH kinase (PI(3)K) was recently implicated in mediation of cardiovascular protective effects of estrogen (Simoncini T. and J. K., 2000). Stimulation with estrogen increases ERα associated PI(3)K activity, thereby leading to the activation of protein kinase B/Akt and endothelial nitric oxide synthase (eNOS).

In neuronal cells, rapid activation of the MAP kinase signaling pathway by E2 results in neuroprotection in primary cortical neurons after glutamate excitotoxicity (Singer et al., 1999). These neuroprotective effects of estrogens, which were reported to occur within 5 minutes after exposure, were mediated through the transient activation of c-Src-tyrosine kinases and tyrosine phosphorylation of p21 (ras)-guanine nucleotide activating protein in an ER-dependent manner.

Similarly, in osteoblasts E2 induces MAP kinase phosphorylation and activation within 5 minutes (Endoh et al., 1997). MAP kinase activation by estrogens may, therefore, regulate cell proliferation and differentiation in these cells leading to increased bone formation. Several studies also indicate that estrogens activate the MAP kinase pathway in osteoclasts through activation of Src kinase (Oursler, 1998). It has been also shown that MAP kinase activation, could be involved in the regulation of enzymes needed for bone resorption (Kristen D. Brubaker, 1999).

In the human mammary cancer derived cell lines, MCF-7 and T47D, as well as in the human colon cancer derived cell line, Caco-2, E2 activates the signal transducing Src/Ras/Erk pathway (Migliaccio et al., 1993), (Migliaccio et al., 1996), (Migliaccio et al., 1998), (Migliaccio et al., 2000). This activation is mediated by direct ER-Src interaction. Interestingly, progesterone also activates the same pathway in T47D cells (Migliaccio et al., 1998). The Src/Ras/ERk signaling pathway is a well-known target of growth factors. Importantly, activation of this pathway requires direct interaction of ER with Src. Activation of this pathway triggers different cellular responses such as proliferation or differentiation [Cantley, 1991]; [Marshall, 1996]; [Downward, 1997]. Its activation by estrogen receptor ligands, such as steroid hormones, explicates their involvement in the cell cycle control.

These data support the view that non-transcriptional/nongenomic activity of ER may be responsible for stimulation of cell growth. However, the details of the molecular mechanism of this process are largely unknown. Src activation has been previously observed in a large number of human breast and colon carcinomas ([Rosen, 1986]; [Ottenhoff-Kalff, 1992]). Activation of Src induces mammary tumors in transgenic mice ([Guy, 1994]). Constitutively active Ras mutants have been found in 25-30% of all cancers, including breast cancer ([Kasid, 1987]).

Recent studies have suggested the existence of a plasma membrane estrogen receptor unrelated to the classical ER. However, cloning or isolation of this membrane ER has not been accomplished, while others have suggested that a subpopulation of the classical ER is associated with the cell membrane and is responsible for the rapid effects of estrogens.

Transcriptional activity of nuclear receptors itself is a target for tight regulation by multiple signaling pathways, including those stimulated by the neurotransmitter dopamine ([Power, 1991]; [Smith, 1993]), growth factors such as epidermal growth factor (EGF), transforming growth factor-α (TGF-a) and insulin-like growth factor I (IGF-I) ([Ignar-Trowbridge, 1996], [Aronica, 1993]; [Bunone, 1996]), and by activators of protein kinase C ([Aronica, 1994]). The molecular mechanisms for such cross coupling are thought to be mediated, at least in part, by receptor phosphorylation. The ER has been demonstrated to be phosphorylated by MAP kinase in response to EGF, thereby resulting in stimulation of AF1 ([Kato, 1996]; [Bunone, 1996]). Other studies have demonstrated that the ER ([Auricchio, 1987]; [Arnold, 1995]; [Pietras, 1995]), Thyroid hormone receptor (TRβ) ([Lin, 1992]), RARγ ([Rochette-Egly, 1992]), glucocorticoid receptor ([Rao, 1987]) and the orphan receptor HNF-4 ([Ktistaki, 1995]) are also targets for tyrosine phosphorylation. A specific tyrosine phosphorylation site has been identified within the AF2 domain of the human ER (hER) at amino acid 537 (Y537) ([Castoria, 1993]; [Arnold, 1995]). This tyrosine is located immediately N-terminal of the AF2 sequence and is conserved in all known ER sequences from diverse species including the ERβ ([Kuiper, 1996]; [Mosselman, 1996]).

Although the molecular mechanisms of non-genomic and genomic activities of E2 are not well understood, the present invention should allow us to create a new generation of therapeutics with tissue and gene selective activity.

To better understand the tissue selective action of some ER ligands, we evaluated the expression and activity of ER-interacting proteins in different cell lines. Using affinity purification and mass spectrometer based microsequencing, we discovered a novel ER interacting protein, designated as MNAR (modulator of non-genomic activity of nuclear receptors).

### SUMMARY OF THE INVENTION

The present invention provides for a novel protein modulator of non-genomic activity of nuclear receptors (MNAR) and nucleic acids that encode such proteins. The MNAR protein can interact with nuclear hormone receptors, such as estrogen receptors. In general, the protein or polypeptide of MNAR refers to the amino acid sequence of an MNAR. In particularly preferred embodiments, MNAR protein interacts with ERα and ERβ. This interaction is ligand dependent. MNAR will also form a complex with ER and kinases, such as members of the Src family. Src and MAP kinase activation leads to enhancement of ER transcriptional activity.

The present invention provides (a) a novel nucleic acid comprising a polynucleotide encoding an MNAR having a putative amino acid sequence of SEQ ID No: 2 (see FIG. 12). A nucleotide sequence of human MNAR is shown in SEQ ID No: 1 (See FIG. 15). An amino acid sequence of human MNAR is shown in SEQ ID NO: 13 (See FIG. 16). A nucleotide sequence of murine MNAR is shown in SEQ ID NO: 12 (See FIG. 17). An amino acid sequence of murine MNAR is shown in SEQ ID NO: 13 (See FIG. 18).

This invention provides novel isolated nucleic acid comprising a polynucleotide selected from the group consisting of:
(a) a polynucleotide encoding a novel protein modulator of non-genomic activity of nuclear receptors (MNAR) comprising the amino acid sequence of SEQ ID NO:2 or of SEQ ID NO:13;
(b) a polynucleotide that hybridizes under highly stringent conditions with (i) a region of the nucleotide sequence of SEQ ID NO: 1 or of SEQ ID NO:12, (ii) a subsequence of at least 100 nucleotides of the nucleotide sequence of SEQ ID NO: 1 or of SEQ ID NO:12, (iii) or a complementary strand of (i) or (ii);
(c) a polynucleotide comprising a sequence with at least 85% identity to a polynucleotide coding sequence of SEQ ID NO: 1 or of SEQ ID NO: 12;
(d) a variant of the polynucleotide comprising a polynucleotide coding sequence of SEQ ID NO:1 or of SEQ ID NO:12; and
(e) a polynucleotide encoding a polypeptide fragment comprising the amino acids of SEQ ID NOS 20 or 23 (f) a complement or antisense form of the polynucleotide of (a), (b), (c), (d) or (e);
   wherein the polynucleotide does not encode a p160 protein (FIG. 1).
   In preferred embodiments the isolated nucleic acid comprises an allelic variant of the polynucleotide.

In certain embodiments, the nucleic acid is operably linked to an expression control sequence (e.g. heterologous or homologous control sequences). This invention provides for a host cell, including bacteria, yeast, insect, and mammalian cells transformed with such isolated nucleic acids.

This invention also relates to a polypeptides comprising an amino acid sequence selected from the group consisting of:
(a) an amino acid sequence for a protein modulator of non-genomic activity of nuclear receptors (MNAR) comprising the amino acid sequence of SEQ ID NO: 2 or of SEQ ID NO:13;
(b) an amino acid sequence for an MNAR fragment comprising the amino acid sequence of SEQ ID NOS:14, 15 or 16;
(c) a variant of (a) or (b), and
(d) a fragment of (a), (b), or (c).
   In alternative embodiments, these polypeptides comprise an amino acid sequences which is more than 90%, or 95% identical to the amino acid sequence of SEQ ID NO:13 or 14. In particular embodiments, the polypeptides of the invention comprise the amino acid sequence of SEQ ID NOS 2, 13, 14, 15 16 20 or 22. In additional embodiments the polypeptides of this invention can stimulates the activity of a tyrosine kinase in the presence of one or more nuclear receptors. The nuclear receptors can be selected from steroid and non-steroid nuclear receptors as well as orphan nuclear receptors. In certain embodiments, the nuclear receptor is an estrogen receptor.

Alternative embodiments involve monoclonal and polyclonal antibodies which correspond to MNAR polypeptides as well as fragments thereof.

In view of the modulation by MNAR of nuclear receptors, many embodiments of this invention relate to modulating the transcriptional activity of a nuclear receptor, comprising providing to the loci of a nuclear receptor an MNAR polypeptide. For example, one can modulate the transcriptional activity of a nuclear receptor, by (a) transforming a host cell with the recombinant vector having the nucleic acid sequence for MNAR according to claim 1; and (b) culturing said transformed host cell to express the nucleic acid in the presence of a nuclear receptor.

This invention also presents numerous approaches for identifying compounds that affect non-genomic activity or genomic activity of nuclear receptors. In one embodiment, a method of identifying compounds exhibiting non-genomic activity versus genomic activity compries administering a test compound to a cell comprising an MNAR-nuclear receptor complex and measuring the non-genomic or genomic activity resulting from the test compound. In these methods, preferably, at least one ligand of a nuclear receptor is present in the cell, or a kinase is present, or both are present. Other methods are directed to the screening for a compound that modulates the activity of MNAR on a nuclear receptor, comprising the steps of (a) contacting a test compound with an MNAR polypeptide; and (b) determining whether said test compound specifically binds said polypeptide. In addition, one can conduct a method of screening for a compound that modulates the activity of MNAR on a nuclear receptor, said method comprising the steps of (a) adding a test compound to a cell comprising the polypeptide of and a nuclear receptor; and (b) comparing the MNAR activity before and after said adding step. Further approaches to this method involve adding a test compound to a control comprising a mutant cell lacking MNAR activity or with significantly reduced MNAR activity. In each of these methods one can use a control to assess the non-genomic activity (or genomic activity) of a compound by administering a compound to a cell in the presence of MNAR and a nuclear receptor, and then repeating the experiment in the absence of MNAR and comparing the level of non-genomic activity. Preferred methods employ a cell overexpresses MNAR a nuclear receptor or both. The genomic activity of a compound is detected by having the nuclear receptor operatively associated with a reporter.

Certain embodiments of the present invention also relate to a method of identifying compounds having selective genomic versus non-genomic activity said method comprising the steps of (a) adding a test compound to a cell comprising a MNAR-nuclear receptor complex; and (b) comparing the genomic versus non-genomic activity before and after said adding the test compound. A selective genomic activity can be measured by conventional means. Preferably, the increase or positive effect of non-genomic activity, as measured, is a two-fold increase after addition of said test compound to a cell in the presence of MNAR-nuclear receptor when compared to genomic activity with test compound in the absence of MNAR and wherein no change is observed in non-genomic activity after addition of said test compound. On the other hand, when selecting for test compounds for non-genomic activity, there should be at least about a two-fold increase after addition of said test compound to a cell in the presence of MNAR-nuclear receptor complex when compared to non-genomic activity with test compound in the absence of MNAR and wherein no change is observed in genomic activity after addition of said test compound. One can measure the effect by determining an increase or decrease in the transcriptional activity of a nuclear receptor in the presence of MNAR.

In a variety of cells, healthy or diseased, it can be valuable to understand whether a beneficial or healthy state for a cell results from non-genomic or genomic activity. The present invention permits such determinations by comparing a desired phenotype of a cell, in the presence and absence of MNAR.

The present invention also relates to non-human transgenic mammals, wherein one or more cells comprise at least one non-functional endogenous MNAR polynucleotide sequence, at least one non-functional endogenous nuclear receptor or both. In preferred embodiments, the non-human transgenic mammal do not have functional endogenous forms of ERa and ERA. Alternatively, transgenic mammal comprises substitution of an inducible/repressable promoter for the endogenous MNAR promoter. The non-human transgenic mammal of claim may further comprise an exogenous selectable marker gene under the control of a promoter active in at least one cell type of said mammal.

This invention also relates to a method of screening for a compound that disrupts the function of MNAR, comprising using a protein-protein interaction assay to identify compounds that interact with MNAR. Once can employ any of the well-known two-hybrid assays or other conventional assays to study protein-protein interactions and the disruption thereof by test compounds, which can be a natural product isolate, small molecule chemical, peptide, etc.

MNAR protein and MNAR encoding nucleic acids can be obtained from mammalian cells and tissues (e.g. human and murine cells and tissues). Recombinant MNAR and anti-MNAR antibodies find use in drug screening, diagnostics, and therapeutics. In particular, the MNAR provides valuable reagents in developing specific biochemical assays for screening compounds for ER ligands that agonize or antagonize the enzymatic activity of the Src family of tyrosine kinases, which are involved in regulating gene expression. Additionally, the MNAR provides valuable reagents in developing specific biochemical assays for screening compounds for a variety of nuclear receptor ligands. Accordingly, one can analyze the effects on any pathway involving a nuclear receptor, such as ER, and/or a kinase. For example, one can use MNAR to set up assays to determine the effect of ER ligands or test compounds on (I) cell cycle stimulation in cancer cells; (II) cell proliferation, cell differentiation and cell conservation for bone development or modulation; (III) neuroprotective activity of ER ligands/test compounds in nerve cells; and (IV) pulmonary characteristics of endothelial cells.

### BRIEF DESCRIPTION OF THE DRAWINGS

FIG. 1 is an alignment comparing the amino acid sequence of MNAR and p160, a protein homologous to MNAR.

FIG. 2 shows estrogen receptor β interaction with MNAR. Panel A presents a silver stained gel obtained by GST-ERβ-LBD pull-down from MCF-7 cells extract. The resulting peptides were purified using reverse phase HPLC column and identified by mass spectrometry based microsequencing. The sequences of these peptides are provided in panel B.

FIG. 3. depicts the results of the expression analysis of MNAR in various cell lines, as evaluated by Northern blotting from experiments in Example 2.

FIG. 4 (A and B) shows the results of MNAR expression analysis in various tissues, as evaluated by Northern blotting from experiments in Example 2.

FIGS. 5A and 5B show analysis of flag-tagged MNAR interaction with ERα and ERβ, or receptors liganded with E2, or 4-OH tamoxifen. Also shown are the results of full-length flag-MNAR interaction with transcribed/translated androgen (AR) and glucocorticoid receptors (GR) with and without their corresponding ligands (FIGS. 5C and 5D).

FIG. 6A depicts the results of the experiment of example 4, wherein the tests are conducted to evaluate the in vitro interaction of Src or Lck with MNAR and/or ERa in the presence and in the absence of 17β-estradiol.

FIG. 6B depicts the results of the experiment of example 4, wherein the tests are conducted to evaluate the in vitro interaction of Src or Lck with MNAR and/or ERβ in the presence and in the absence of 17β-estradiol.

FIG. 7 is a schematic depiction of the proposed model of ER-MNAR-Src interaction.

FIG. 8 is an SDS-PAGE gel analysis of the ER-MNARc complex on cSrc enzymatic activity. cSrc enzymatic activity was evaluated in the absence (lanes 1 and 2) or presence of ER (lanes 3-6), E2 (lanes 3 and 5), and MNAR (lanes 2, 5, and 6) using acid denatured enolase as a substrate. Data presented in FIG. 8 indicate that MNAR itself strongly stimulates cSrc enzymatic activity.

FIG. 9 is a series of graphs depicting the results from the experiments of example 7, wherein the tests are conducted to evaluate whether MNAR affects ER-mediated transcription. FIG. 9A shows the result for ERα, MNAR, and/or SRC3. FIG. 9B depicts the results for ERα and MNAR when cells are treated with E2, E2 plus PP2, or E2 plus PD98059. FIG. 9C shows results of MCF-7 cells treated with antisense (AS), E2, E2 plus control, or E2 plus antisense. A primer/probe set is designed to target MNAR, PS2, and cathepsin D, respectively. FIG. 9D shows results of a Western blot analysis of MCF-7 cell extracts transfected with antisense or reverse control oligomers stimulated with E2 with rabbit polyclonal MNAR antiserum.

FIG. 10 depicts an SDS-gel analysis of MCF-7 cells untransfected and/or transfected with a Flag-MNAR expression vector untreated and treated with ±2. Flag-MNAR (panel A), ERα (panel C and D), and cSrc (panel B) were immunoprecipitated from the cell extracts using their corresponding antibodies. Material precipitated using ERα antiserum was probed with rabbit polyclonal anti-MNAR antibody (panel E). E2-enhancement of ERα-MNAR interaction was clearly detected.

FIG. 11 depicts the results of the analysis of MNAR on E2 induced Erk activation, as evaluated by Western blotting from experiments in Example 9.

FIG. 12 is a schematic depiction of the structural-functional organization of MNAR and 13 MNAR mutants (mutants 2-14), which were generated and tested for their ability to enhance ER-stimulated transcription when transfected into HepG2 cells under the experiments of Example 10.

FIG. 13 is a bar graph depicting the results of an analysis of MNAR deletion mutants (mutants 2-14), which were tested for their ability to enhance ER-stimulated transcription when transfected into hepG2 cells in the experiments of Example 10.

FIG. 14 depicts is a bar graph depicting the results of an analysis of PXXP motif MNAR mutants, which were tested for their ability to activate ER transcriptional activity When transfected into HepG2 cells in the experiments of Example 10.

FIG. 15 depicts a polynucleotide sequence of human MNAR (SEQ ID NO:1).

FIG. 16 depicts an amino acid sequence of human MNAR (SEQ ID NO: 2).

FIG. 17 depicts a polynucleotide sequence of murine MNAR (SEQ ID NO:12)).

FIG. 18 depicts an amino acid sequence of murine MNAR (SEQ ID NO:13).

### DESCRIPTION OF THE SPECIFIC EMBODIMENTS

Definitions

Functional analogs of a given MNAR protein or protein with “substantial functional identity” to a given MNAR are defined as proteins that exhibit one or more biochemical properties specific to such MNAR.

“Nucleic acid molecule” means a single-stranded or double stranded molecule and includes RNA molecules, DNA molecules, and analogs of RNA or DNA molecules.

“Modulating transcription” means altering transcription, and includes changing the rate of transcription initiation, the level of transcription, or the responsiveness of transcription/transcription initiation to regulatory controls.

“Neoplastic state of cells” means any new growth of cells which may be benign or malignant.

The terms “substantially pure” or “isolated” mean that the MNAR protein, MNAR protein fragment, or nucleic acid encoding a MNAR or MNAR fragment are unaccompanied by at least some of the material with which it is normally associated in its natural state. A composition of a substantially pure MNAR or portion thereof may contain excipients and additives useful in diagnostic, therapeutic and investigative reagents.

“Altered expression state” means that the expression of MNAR in a naturally-occurring state is altered to increase or decrease MNAR expression. In comparison to the naturally-occurring state.

“Substantial sequence identity” means that a portion of the protein or nucleic acid presents at least about 70%, more preferably at least about 80%, and most preferably at least about 90%, 95%, or 99% sequence identity with a MNAR sequence portion. Where the sequence diverges from native MNAR sequences disclosed herein, the differences are preferably conservative, i.e., an acidic for an acidic amino acid substitution or a nucleotide change providing a redundant codon. Dissimilar sequences are typically aggregated within regions rather than being distributed evenly over the polymer. A substantially identical sequence hybridizes to a complementary MNAR-encoding sequence under relatively high stringency. For the purposes of defining high stringency southern hybridization conditions, reference can conveniently be made to Sambrook et al. (1989) at pp. 387-389 which is herein incorporated by reference where the washing step at paragraph 11 is considered high stringency.

“Fusion protein or polypeptide” means a protein or polypeptide that comprises at least a first amino acid sequence fused to a second amino acid sequence, where such fusion does not occur in a naturally occurring protein or polypeptide. Preferably, at least one amino acid sequence in the fusion protein or polypeptide is that of MNAR.

“Mutant or mutation” means any detectable change in genetic material, e.g. DNA, or any process, mechanism or result of such a change. This includes gene mutations, in which the structure (e.g. DNA sequence) of a gene is altered, any gene or DNA arising from any mutation process, and any expression product (e.g. protein, polypeptide, or enzyme) expressed by a modified gene or DNA sequence.

“Variant” is used to indicate a modified or altered gene, DNA sequence, protein, polypeptide, enzyme, cell, etc., i.e. any kind of mutant. A variant of a naturally occurring protein or polypeptide, for example, comprises a substitution, deletion, modification, and/or insertion of one or more amino acids in the naturally occurring protein or polypeptide.

“Recombinant” means any genetic material (e.g. DNA sequence) resulting from the insertion into the sequence or chain, by biological or chemical means, of a sequence (e.g. a full-length or partial DNA sequence) not originally biologically or naturally present in that genetic material (e.g. DNA sequence).

“Protein or polypeptide fragment” means any smaller portion of a larger protein or polypeptide, where preferably the fragment has at least 8 amino acids and more preferably, 25, 50, 100 or 200 amino acids.

Non-genomic activity of a receptor is “beneficial to a cell” wherein the cell exhibits presence of a desired activity or phenotype upon introduction of increased amount of MNAR to the cell.

Non-genomic activity of a receptor is “not beneficial to a cell” wherein the cell exhibits absence of a desired activity or phenotype upon introduction of increased amount of MNAR to the cell.

MNAR Characterization

Sequence analysis MNAR revealed multiple LXXLL motifs localized in N-terminal portion of MNAR′ molecule. Similar motifs in other transcription factors were previously shown to form contacts with a hydrophobic groove on the surface of the ligand binding domains of nuclear receptors ((Heery et al., 1997), (Torchia et al., 1997)). Another important feature of the MNAR molecule is a proline-rich domain. This domain can be utilized for interaction with SH3 domains of the Src family of protein kinases. Initially we hypothesized that MNAR may potentiate nuclear receptors interaction with Src kinases. Our data indicate that estrogen receptors α and β interact with MNAR, and this interaction is enhanced by 17β-estradiol but not by 4(OH) tamoxifen. Interestingly, the MNAR-ER complex, both in-vitro and in-vivo strongly interacts and stimulates enzymatic activity of Src kinases. Importantly, overexpression of MNAR leads to enhancement of ER transcriptional activity. We expect that this activation of ER is due to its phosphorylation by one of the kinases downstream of Src. Accordingly, we submit that MNAR modulates ER interaction with Src kinases and by doing so mediates the nongenomic activity of ER.

Our data supports the development of experimental models that will allow us to assay for ER ligands that are able to differentiate between genomic and non-genomic activity of ER ligands. Ligands that do not affect transcriptional activity of ER, but stimulate the Src enzymatic activity may possess important bone-sparing, CNS protective, and cardioprotective activity. On the other hand, compounds that selectively control transcriptional activity of ER would have a much more selective action, thereby avoiding multiple side affects. Overall, our ability to separate genomic and nongenomic activity of nuclear receptor ligands, particularly ER ligands, may allow us to create a new generation of therapeutics with profoundly focused, selective action.

Our data also support the development of experimental models that will allow us to assay for nuclear receptor ligands that are able to differentiate between genomic and non-genomic activity of nuclear receptor ligands. Ligands that do not affect transcriptional activity of various nuclear receptors, but stimulate the Src enzymatic activity or other non-genomic enzymatic activity may possess important therapeutic properties or activities. On the other hand, compounds that selectively control transcriptional activity of nuclear receptors would have a much more selective action, thereby avoiding multiple side affects. Overall, our ability to separate genomic and non-genomic activity of nuclear receptor ligands may allow us to create a new generation of therapeutics with profoundly focused, selective action.

Nucleic Acid Sequences Encoding MNAR and Corresponding MNAR Proteins

A biologically active MNAR, or MNAR fragment thereof, retains one or more of the MNAR's functions, such as the ability to specifically form a complex with ER, or to modulate or enhance the transcriptional activity of ER. Exemplary assays for biological activity are described below and in the working exemplification. Specific binding is empirically determined by contacting, for example a MNAR, with a mixture of components and identifying those components that preferentially bind the MNAR. Specific binding may be conveniently shown by a number of methods, including but not limited to yeast and mammalian hybrid systems and competitive binding studies. For example, a human cDNA library was screened using the yeast two-hybrid assay to identify potential coactivators, receptors and/or ligands for ER or MNAR.

In addition to the analysis of the interactions between kinases and the MNAR-ER complex, MNAR can be used in similar strategies to assess the interaction between ER ligands and ER with other known transcription factors, including other nuclear receptors, and to identify novel interacting proteins. For example, since the MNAR-ER interaction can be enhanced by 17β-estradiol, one can assess the effect of test agents, which may be compounds or proteins or peptides. Furthermore, the yeast two-hybrid system, the mammalian two-hybrid system, surface plasmon resonance assays, immunoprecipitation assays, or other assays can be used to demonstrate a direct interaction between MNAR and ER in the presence or absence of Src. Similarly, these assay systems could be used to broadly screen for known and novel proteins that interact with MNAR-ER complex or MNAR-ER-Src complex.

The invention provides recombinantly produced MNAR proteins, MNAR analogs and fragments thereof. These recombinant products are readily modified through physical, chemical, and molecular techniques disclosed or cited herein or otherwise known to those skilled in the relevant art. According to a particular embodiment of the invention, fragments of the MNAR-encoding sequences are spliced with heterologous sequences to produce fusion proteins. Such fusion proteins find particular use in modulating ER activity in vitro and in vivo.

MNAR can be further modified by methods known in the art. For example, MNAR may be phosphorylated or dephosphorylated, glycosylated or deglycosylated, with or without radioactive labeling, etc. Serine, threonine, and tyrosine residues can particularly provide useful phosphorylation sites. Especially useful are modifications that alter MNAR solubility, membrane transportability, stability, and binding specificity and affinity. Some examples include fatty acid-acylation, proteolysis, and mutations in nuclear receptor transcription factor interaction domains that stabilize binding. Particularly, one may modify the proline-rich region of one of the LXXLL motifs.

A substantially pure or isolated MNAR protein, or MNAR portion encoded by nucleic acid, is generally at least about 1% homologous to said MNAR encoding nucleic acid; preferably at least about 10%; more preferably at least about 50%; and most preferably at least 90%. Nucleic acid weight percentages are determined by dividing the weight of the MNAR or MNAR portion encoding nucleic acid, including alternative forms and analogs such as alternatively spliced or partially transcribed forms, by the total nucleic acid weight present.

The invention also provides for MNAR sequences modified by transitions, transversions, deletions, insertions, or other modifications such as alternative splicing and such alternative forms, genomic MNAR sequences, MNAR gene flanking sequences, including MNAR regulatory sequences and other non-transcribed MNAR sequences, MNAR mRNA sequences, and RNA and DNA antisense sequences complementary to MNAR encoding sequences, sequences encoding xenogeneic MNARs and MNAR sequences comprising synthetic nucleotides, e.g., the oxygen of phosphate group may be replaced with sulfur, methyl, or the like.

The modified MNAR encoding sequences or related sequences encode proteins with MNAR-like functions. There will generally be substantial sequence identity between at least a portion thereof and a portion of a MNAR, preferably at least about 40%, more preferably at least 80%, most preferably at least 90%, 95%, or 99%, particularly conservative substitutions, particularly within the proline-rich regions and LXXLL motifs and regions encoding protein domains involved in protein-protein interactions, particularly MNAR-ER or MNAR-ER-kinase interactions, particularly MNAR-nuclear receptor or MNAR-nuclear receptor-kinase interactions.

MNAR can be subject to alternative purification, biosynthesis, modification or use by methods disclosed herein or otherwise known in the art. For example, the amino acids can be modified in a number of ways, including but not limited to, altering stability, solubility, binding affinity, specificity, and methylation. The amino acid sequences of the present invention may also be modified with a label capable of providing a detectable signal, either directly or indirectly. Exemplary labels include, but are not limited to, radioisotopes, fluorescers, and biotinylation.

Nucleic acids encoding at least a portion of an MNAR are used to identify nuclear factors and agents which interact with that MNAR using expression screening in yeast or mammalian cells as described in Current Protocols in Molecular Biology (See “Protein Expression” in Chapter 16, 2002). One method of identifying these nuclear factors and agents, which interact with MNAR, is to utilize a mammalian, or yeast, two-hybrid or three-hybrid assay. (See, e.g., Fields S. et al., (1994) Trends Genet. 10:286-292; Young P. et al. (1992) Current Biology 3:408-420; Zervos et al. (1993) Cell 72:223-232; Madura et al. (1993) J. Biol. Chem. 268:12046-12054; Bartel et al (1993) Biotechniques 14:920-924; and Iwabuchi et al. (1993) Oncogene 8:1693-1696). As exemplified herein, a yeast cDNA library containing fusion genes of cDNA joined with DNA encoding the activation domain of a transcription factor (e.g. Gal4) are transfected with fusion genes encoding a portion of a MNAR and the DNA binding domain of a transcription factor. Clones encoding MNAR binding proteins provide for the complementation of the transcription factor and are identified through transcription of a reporter gene. See, e.g. Fields and Song (1989) Nature 340, 245-246 and Chien et al. (1991) Proc. Natl. Acad. Sci USA 88, 9578-9582.

The invention also provides vectors comprising nucleic acids encoding MNAR or portion or analog thereof and optionally, encoding ERs and or kinases. A large number of vectors, including plasmid and viral vectors, have been described for expression in a variety of eukaryotic and prokaryotic hosts. Vectors will often include one or more replication systems for cloning or expression, one or more markers for selection in the host, e.g., antibiotic resistance, and one or more expression cassettes. The inserted MNAR coding sequences may be synthesized, isolated form natural sources, prepared as hybrids, etc. Ligation of the coding sequences to the transcriptional regulatory sequences may be achieved by known methods. Advantageously, vectors may also include a promoter operably linked to the MNAR encoding portion.

Suitable host cells may be transformed/transfected/infected by any suitable method including electroporation, CaCl₂ mediated DNA uptake, viral infection, microinjection, microprojectile, or other established methods. Alternatively, nucleic acids encoding one or more MNARs may be introduced into cells by recombination events. For example, a sequence can be microinjected into a cell, and thereby effect homologous recombination at the site of an endogenous gene encoding a MNAR, an analog or pseudogene thereof, or a sequence with substantial identity to a MNAR-encoding gene. Other recombination based methods such as non-homologous recombinations, deletion of endogenous gene by homologous recombination, especially in pluripotent cells, etc., provide additional applications.

Appropriate host cells include bacteria, archebacteria, fungi, especially yeast, and plant and animal cells, especially mammalian cells. Of particular interest are E. coli, B. subtilis, Saccharomyces cerevisiae, A549 cells, CHO, COS, HeLa, Hep2, and bone cells and immortalized mammalian myeloid and lymphoid cell lines. Preferred host cell lines include but are not limited to human cancer cell lines such as MCF-7 and T47D cells. Ideally, such expression systems utilize inducible expression strategies like the TET ON/OFF system that is commercially available. Such cell lines are useful to define the role of MNAR in non-genomic ER activity and to assess the effect of potential compounds that modulate MNAR-ER interactions. A large number of transcription initiation and termination regulatory elements/regions have been isolated and shown to be effective in the transcription and translation of heterologous proteins in the various hosts. Examples of these regions, methods of isolation, manner of manipulation, etc. are known in the art. Suitable expression control sequences and host cell/cloning vehicle combinations are well known, and are described in Sambrook et al. (1989).

MNAR encoding oligonucleotides can also be used to identify other MNARs or transcription factor coactivators. For example, ³²P-labeled MNAR encoding nucleic acids are used to screen cDNA libraries at low stringency to identify similar cDNAs that encode proteins with MNAR related domains. Additionally, MNAR related proteins are isolated by PCR amplification with degenerate oligonucleotide probes using the sequences disclosed herein. Other experimental methods for cloning MNAR are also set out in the working exemplification below. Other useful cloning, expression, and genetic manipulation techniques for practicing the inventions disclosed herein are known to those skilled in the art.

The compositions and methods disclosed herein may be used to effect gene therapy. See, e.g., Gutierrez et al. (1992) Lancet 339, 715-721. For example, cells are transfected with MNAR sequences operably linked to gene regulatory sequences capable of effecting altered MNAR expression or regulation. To modulate MNAR translation, cells may be transfected with MNAR complementary antisense polynucleotide.

Antisense modulation may employ MNAR antisense sequences operably linked to gene regulatory sequences. An antisense oligonucleotide can be, for example, 8, 10, 15, 20, 25, 30, 35, 40, 45 or 50 nucleotide sequences in length. An antisense nucleic acid can be constructed using chemical synthesis and enzymatic ligation reactions using known procedures in the art. For example, an antisense nucleic acid (e.g. antisense oligonucleotide) can be chemically synthesized using naturally occurring nucleotides or various modified nucleotides designed to increase the biological activity of the molecules or to increase the physical stability of the duplex formed between antisense and sense nucleic acids, e.g. phosphorothiolate derivatives and acridine substituted nucleotides can be used. Alternatively, the antisense nucleic acid can be produced biologically using an expression vector into which a nucleic acid has been subcloned in an antisense orientation (i.e. RNA transcribed from the inserted nucleic acid will be of an antisense orientation to a target nucleic acid of interest. In the present invention, cells are transfected with a vector comprising a MNAR sequence with a promoter sequence oriented such that transcription of the gene yields an antisense transcript capable of binding to MNAR encoding mRNA. Transcription may be constitutive or inducible and the vector may provide for stable extrachromosomal maintenance or integration. Alternatively, single-stranded antisense nucleic acid sequences that bind to genomic DNA or mRNA encoding at least a portion of MNAR may be administered to the target cell at a concentration that results in a substantial reduction in MNAR expression.

Assays for Identifying Modulators of Non-Genomic Activity of Nuclear Receptors and Therapeutic Agents

The invention provides methods and compositions for identifying agents useful in modulating the genomic or non-genomic activity of nuclear receptors (e.g. ER). Such agents find use in the diagnosis or treatment of broad range of disease including, but not limited to, cancer, cardiovascular diseases, microbial and fungal infections and particularly immune disease, bone protection, etc. The ability to develop rapid and convenient high-throughput biochemical assays for screening compounds that work through nuclear receptor (e.g. ER) non-genomic activity versus nuclear receptor (e.g. ER) genomic activity in human cells opens a new avenue for drug development. In addition, this invention provides a tool for identifying whether disease states within a cell or beneficial cell states of a cell are effected by nuclear receptor (e.g. ER) genomic/non-genomic activities/pathways, i.e. one can determine whether cancer cells proliferate when nuclear receptor (e.g. ER) non-genomic activity or nuclear receptor (e.g. ER) genomic activity is decreased/enhanced. In general, this invention provides for categorizing many cell types as requiring nuclear receptor (e.g. ER) genomic/non-genomic activity.

Common assays include yeast two-hybrid assays as disclosed by Young and Ozenberger in U.S. Pat. No. 5,989,808, which is herein incorporated by reference. The two hybrid assays can be used to screen for small molecule compounds or peptides or proteins of interest.

Typically, prospective agents are screened from large libraries of synthetic or natural compounds. For example, numerous means are available for random and directed synthesis of saccharide, peptide, and nucleic acid based compounds, see, e.g. Lam et al. (1991) Nature 354, 82-86. Alternatively, libraries and compounds are readily modified through conventional chemical, physical, and biochemical means. Examples of such modifications are disclosed herein.

Useful agents are identified with a range of assays employing MNARs or MNAR encoding nucleic acids. The assays may also employ one or more selected from nuclear receptors, steroid receptors, orphan receptors, ER, ERE (estrogen response elements), ER ligands, kinases and inhibitors of such kinases. As examples, protein binding assays, nucleic acid binding assays and gel shift assays are useful approaches. Exemplary assays include assaying labeled nuclear receptor (e.g. ER) binding to immobilized MNAR, labeled MNAR or MNAR peptide binding to immobilized nuclear receptor (e.g. ER), etc. Many appropriate assays are amenable to scaled-up, high throughput usage suitable for volume drug screening. The particular assay used will be determined by the particular nature of the MNAR interactions. Assays may employ a single MNAR, MNAR fragments, MNAR fusion products, partial MNAR complexes, or the complete basal transcription complex comprising an MNAR nucleic acid.

Useful agents are typically those that bind to or modify the association of MNAR, the MNAR-nuclear receptor complex, or the MNAR-ER complex. Preferred agents include those capable of modulating the expression of MNAR genes, particularly genes transcribed by members of the nuclear receptor superfamily.

Useful agents are found within numerous chemical classes, though typically they are organic compounds; preferably small organic compounds. Small organic compounds have a molecular weight of more than 50 yet less than about 2,500, preferably less than about 750, more preferably, less than about 250. Exemplary classes include peptides, saccharides, steroids, and the like.

Selected agents may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such a using an unnatural amino acid, such as a D-amino acid, particularly D-alanine, by functionalizing the amino or carboxyl terminus, e.g., for the amino group, acylation or alkylation, and for the carboxyl group, esterification or amidification, or the like. Other methods of stabilization may include encapsulation, for example, in liposomes, etc.

Agents may be prepared in a variety of ways known to those skilled in the art. For example, peptides under about 60 amino acids can be readily synthesized today using conventional commercially available automatic synthesizers. Alternatively, peptide (and protein and nucleic acid agents) are readily produced by known recombinant technologies.

These assay systems could also be used to screen for potential therapeutics, including peptide and chemical ligands. Such potential therapeutics, activators or inhibitors of MNAR function, could act by either modulating an interaction between MNAR and either a nuclear receptor (e.g. ER) or a kinase (“class I”) or by modulating an activity of MNAR or a nuclear receptor (e.g. ER) or kinase (“class II”). Interaction assays, including but not limited to two-hybrid assays, immunoprecipitation assays, and surface plasmon resonance (SPR) assays, could be used to identify “class I” therapeutics. For example, chemical compounds or peptides can be screened for their ability to modulate the interaction between MNAR and a nuclear receptor (e.g. ER) by SPR. Activity assays, including but not limited to mammalian transfection assays in which nuclear receptor (e.g. ER) transcriptional activity is observed, could be used to identify “class II” compounds. For example, compounds could be screened for their ability to modulate the enhancement of nuclear receptor (e.g. ER) activity by MNAR. Such compounds may modulate the interactions of MNAR with a nuclear receptor (e.g. ER) or they may modulate a known or unknown activity of MNAR or a nuclear receptor (e.g. ER), including a transcriptional activity or assays that can be used to evaluate enzymatic activity of kinases.

For therapeutic uses, the compositions and selected agents disclosed herein may be administered by any convenient way that will depend upon the nature of the compound. For small molecular weight agents, oral administration is preferred and enteric coatings may be indicated where the compound is not expected to retain after exposure to the stomach environment. Generally the amount administered will be empirically determined by routine dose-response experiments. Typically amounts range from about 1 to 1000 μg/kg of recipient. Large proteins are preferably administered parenterally, conveniently in a physiologically acceptable carrier, e.g., phosphate buffered saline, saline, deionized water, or the like. Typically, such compositions are added to a retained physiological fluid such as blood or synovial fluid. Other additives may be included, such as stabilizers, bactericides, etc. These additives will be present in conventional amounts.

MNAR may also be modified with a label capable of providing a detectable signal either directly or indirectly. Exemplary labels include radioisotopes, fluorescers, etc. Alternatively, a MNAR may be expressed in the presence of a labeled amino acid such as ³⁵S-methionine. Such labeled MNAR and analogs thereof find use, for example, as probes in expression screening assays for proteins that interact with MNAR, or, for example, MNAR binding to ER or other ER interacting proteins or transcription factors in drug screening assays.

Specific polyclonal or monoclonal antibodies that can distinguish MNAR from other nuclear proteins are readily made using the methods and compositions disclosed in Harlow and Lane, Antibodies, A Laboratory Manual, Cold Spring Harbor Laboratory, 1988, other references cited herein, as well as immunological and hybridoma technologies known to those in the art. In particular, MNAR and analogs and fragments thereof also find use in raising anti-MNAR antibodies in laboratory animals such as mice and rabbits as well as the production of monoclonal antibodies by cell fusion or transformation.

Anti-MNAR antibodies and fragments (Fab, etc.) thereof find use in modulating MNAR role in transcription complexes, screening MNAR expression libraries, etc. In addition, these antibodies can be used to identify, isolate, and purify structural analogs of MNAR. Anti-MNAR antibodies also find use for subcellular localization of MNAR under various conditions such as infection, during various cell cycle phases, induction with cytokines, protein kinases such as C and A, etc. Other exemplary applications include using MNAR-specific antibodies (including monoclonal or MNAR-derived peptide specific antibodies) to immuno-deplete in vitro transcription extracts and using immuno-affinity chromatography to purify MNAR, including analogs, or other nuclear factors which interact with MNAR.

Compositions are also provided for therapeutic intervention in disease, for example, by modifying MNARs or MNAR encoding nucleic acids. Oligopeptides can be synthesized in pure form and can find many uses in diagnosis and therapy. These polypeptides can be used, for example, to modulate native MNAR interaction with MNAR interacting proteins. The oligopeptides will generally be more than six and fewer than about 60 amino acids, more usually fewer than about 30 amino acids, although large oligopeptides may be employed. A MNAR or a portion thereof may be used in purified form, generally greater than about 50%, usually greater than about 90% pure. Methods for purifying such peptides to such purities include various forms of chromatographic, chemical, and electrophoretic separations disclosed herein or otherwise known to those skilled in the art.

Transgenic Animals and Cells

Animal models serve as useful vehicles for screening of compounds, for example, for effects on cholesterol levels, cholesterol absorption, and bile acid synthesis. Transgenic animals, defined as animals with engineered genomes, may prove particularly useful in certain applications. In the present application, the focus is on the modulation of ER activity, or other nuclear receptors and the role of MNAR in effecting the non-genomic activity of ER. Thus, embodiments of this invention include transgenic mice that lack MNAR activity as well as transgenic animals that may lack nuclear receptor activity, ER activity, or a kinase activity. Animals lacking one of the above are useful to determine the specificity of a compound or ligand for MNAR, ER, a kinase. These animals are useful not only in elucidating the biology of this signaling pathway, but as tools in examining possible therapeutic compositions and methods of treatment.

For these reasons, transgenic non-human animals of the present invention will have at least one non-functional endogenous MNAR (and/or ER and/or kinase). It further is contemplated that the transgenic non-human animal of the present invention may have both MNAR and ER altered, thus providing cells which lack functional MNAR-ER complex. The non-functional attribute(s) can contain an interruption of the gene coding sequence, a nonsense mutation that truncates the protein product, a deletion of the gene coding sequence or alterations in the MNAR regulatory region. In designing a heterologous gene for expression in animals, sequences which interfere with the efficacy of gene expression, such as coding sequences, promoters, introns, polyadenylation signals, polymerase II termination sequences, hairpins, consensus splice sites and the like, may be eliminated.

Current advances in transgenic approaches and techniques have permitted the manipulation of a variety of animal genomes via gene addition, gene deletion, or gene modifications (Franz et al., 1997). The transgenic synthesis of human hemoglobin (U.S. Pat. No. 5,602,306, specifically incorporated herein by reference) and fibrinogen (U.S. Pat. No. 5,639,940, specifically incorporated herein by reference) in non-human animals have also been disclosed, each specifically incorporated herein by reference in it entirety. The construction of a transgenic mouse model has recently been used to assay potential treatments for Alzheimer's disease (U.S. Pat. No. 5,720,936, specifically incorporated herein by reference in its entirety). It is contemplated in the present invention that transgenic animals contribute valuable information as models for studying the effects of ligands specific for the MNAR and/or ER in transgenic MNAR and ER knockout animal models.

### Conclusions

This invention is based on the molecular cloning, structural analysis and characterization of a protein modulator of non-genomic activity of nuclear receptors (MNAR). The present invention demonstrates that human MNAR interacted with nuclear hormone receptors to form a complex and affect the enzymatic activity of Src protein kinases.

Crosstalk between cell surface form of ER and intracellular signaling pathways was first demonstrated more than 20 years ago (Pietras, 1975), (Pietras, 1977). Since then multiple reports confirmed rapid estrogen effects occurring in the vasculature, breast, bone, uterus and neuronal tissues that cannot be explained by the “genomic” action of estrogen receptor. It became clear that biological activity of estrogen in responsive cells is mediated not only through a specific high-affinity receptor, located exclusively in their nuclei, but also through the interaction of estrogen with cell-surface receptor. The nature of this receptor is debated. Some evidence supports the existence of a unique membrane receptor, other favors the identity or the strong similarity between the cell-surface and nuclear estrogen receptors (Pietras and Szego, 1979), (Bression et al., 1986), (Pappas et al., 1995).

Important evidence, based on experiments with COS cells transfected with ERa cDNA, clearly demonstrated that classical estrogen receptor can activate cSrc in the process leading to activation of tyrosine phosphorylation/p21^{ras}/MAP kinase pathway (Migliaccio et al., 1996).

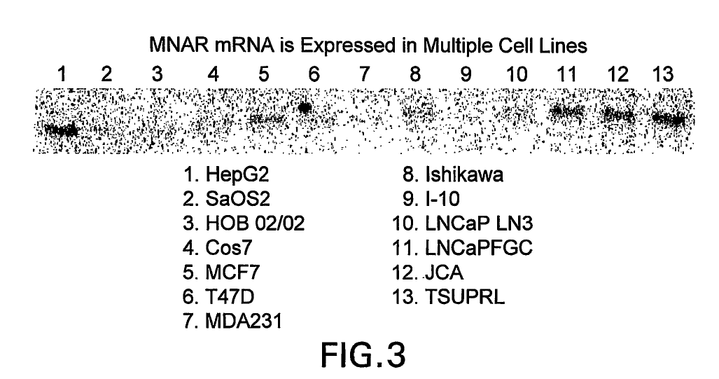

Evaluating the spectrum of ER interacting proteins in MCF7 cells, we discovered a novel ER-interacting protein—MNAR. We showed that both ERα and ERβ interacted with MNAR and affinity of this interaction is enhanced by 17β-estradiol and reduced by 4(OH) tamoxifen (FIG. 3).

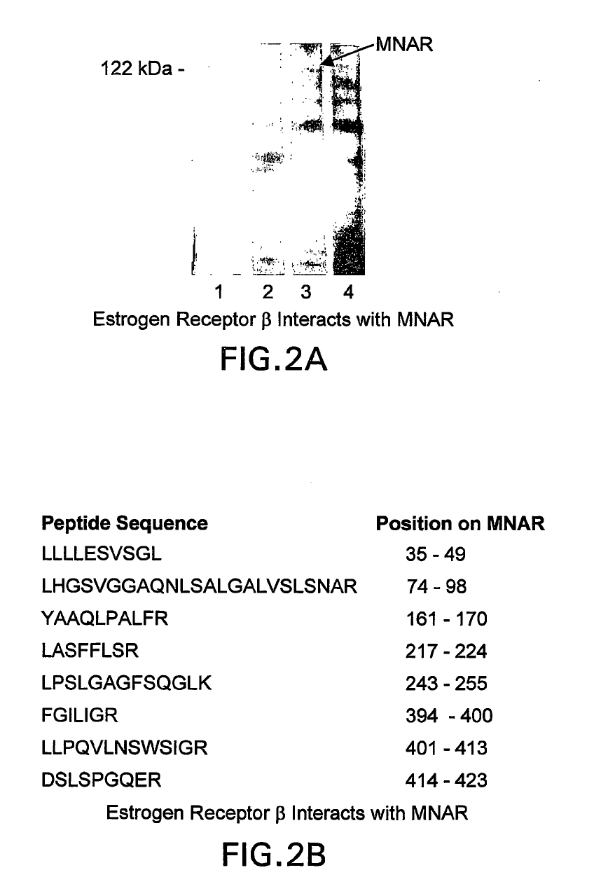

Protein homologous to MNAR—p160 was recently isolated using pull-down approach with SH2 domain of Lck. Our data indicate that MNAR is the major form of this protein expressed in MCF7 cells (FIG. 2).

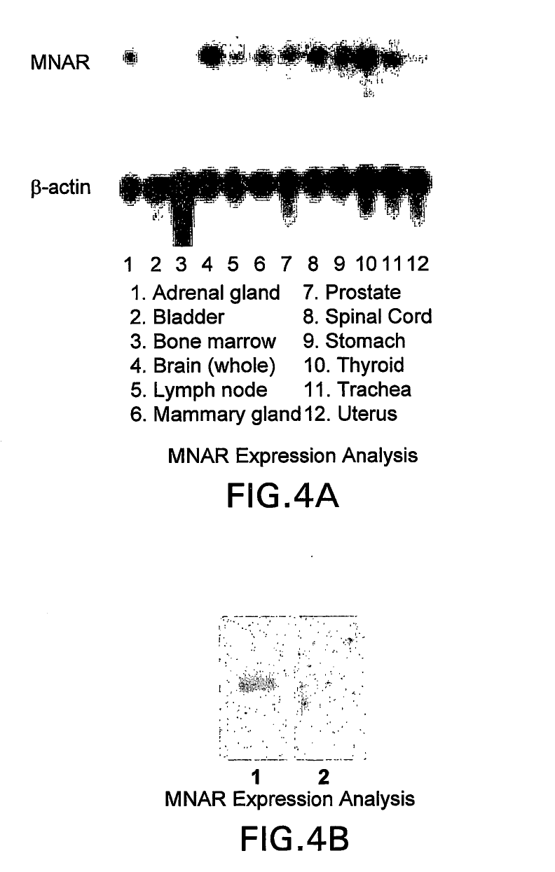

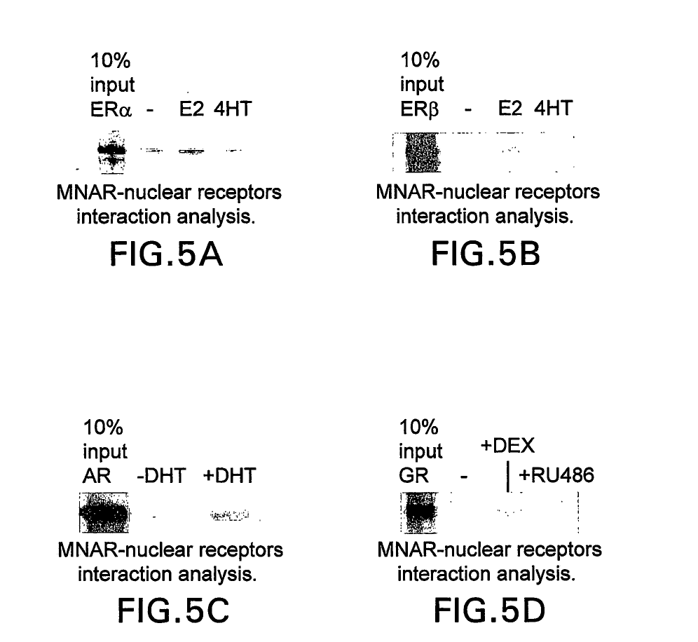

Next we evaluated whether MNAR affects estrogen receptors interaction with Src family kinases. Our data indicate that both ERα and ERβ interact with partially purified cSrc and this interaction is not enhanced by 17β-estradiol (FIGS. 4A and B). This result does not contradict previously reported data (Migliaccio et al., 2000), (Migliaccio et al., 1998) since we used in vitro transcribed/translated ER and MNAR and also purified cSrc. At the same time Migliaccio et al. (Migliaccio et al., 2000), (Migliaccio et al., 1998) performed their experiment by pulling down Src- or ER-interacting proteins from cell extracts that may contain MNAR. Importantly, in presence of MNAR, estrogen receptors interact with cSrc and Lck in ligand-dependent way (FIGS. 4A and B). We also observed that MNAR itself binds to cSrc or Lck with low affinity. Our results therefore suggest that ER and MNAR synergistically bind to Src family kinases. Under basal conditions, the catalytic domain of Src family kinases is constrained in an inactive state through intermolecular interactions. Binding of the SH2 domain to the C-terminal phosphorylated tyrosine locks the molecule in an inhibited conformation ([Matsuda, 1990]). Binding of the SH3 domain to the proline-reach domain of the linker region is also important for kinase inactivation ([Superti-Furga, 1993]). Full catalytic activation requires release of these restrains. The kinase activity of Src can be enhanced by binding of the SH2 domain to phosphotyrosine-containing sequences and by binding of the SH3 domain to the proline-rich sequences ([Hubbard, 1998]). We next investigated if ER-MNAR binding leads to cSrc activation. Our results suggest that MNAR strongly enhances cSrc catalytic activity that can be further stimulated by addition of ER-E2 (FIG. 5). Phosphotyrosine 537 of ERα has been identified as the residue essential for ER interaction with SH2 domain of Src ((Migliaccio et al., 1998)). We submit that ER interacts with LXXLL motifs of the MNAR molecule and ER's phosphotyrosine 537 interacts with SH2 domain of Src. At the same time proline-rich domain of MNAR probably interacts with SH3 domain of Src. Formation of this complex leads to cSrc activation.

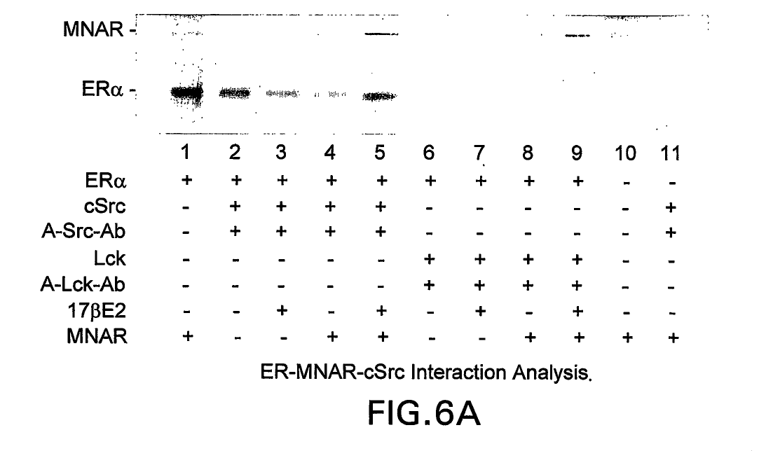

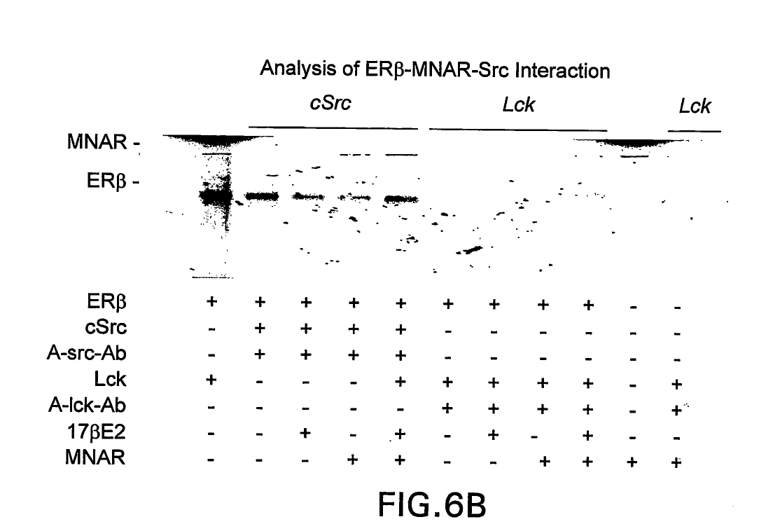

Activation of cSrc in response to EGF has been previously shown to promote the ER phosphorylation by the MAP kinase and stimulation of ER-mediated transcription ([Kato, 1996]; [Bunone, 1996]). Our results indicate that overexpression of MNAR, presumably through activation of cSrc-mediated phosphorylation cascade, augments ER transcriptional activity (FIG. 6A). Attenuation of MNAR-induced ER activation in the presence of Src inhibitors PP2 and genistein (FIG. 6B) supports the view that MNAR interacts with ER via the cSrc-mediated phosphorylation cascade.

Our studies describe a new ER-interacting protein that modulates the cross talk between ER and Src family tyrosine kinases, thus regulating the nongenomic activity of estrogens and potentially other steroids. The nongenomic activity represent an important mechanism by which estrogens regulates cell grows and proliferation. MNAR therefore, may represent an important target for development of new estrogens with more selective action.

The above conclusions are supported by the following examples, which are offered by way of illustration and not by way of limitation.

### EXAMPLES

Equipment and Reagents. 17β-estradiol (E2) and 4(OH) tamoxifen were obtained from Sigma. ICI-182,780 was provided by AstraZeneca Pharmaceuticals (Wlimington. DE). 5′RACE primers were from marathon cDNA kit (BD Biosciences Clontech, Palo Alto, Calif.). Purified cSrc or Lck were from Upstate (Charlottesville, Va.). Biotinylated peptide derived from p34^{cdc2} was synthesized by the Peptide Chemistry group at Wyeth Research. Glutathione agarose beads were obtained from Sigma-Aldrich (St. Louis, Mo.). Anti-phosphotyrosine antibody and SuperSignal Elisa Pico peroxidase substrate were from Perbio Science AB (Pierce, Rockford, Ill.). Src inhibitor PP2 was from Calbiochem-Novobiochem Corporation (San Diego, Calif.).

Method 1:

Isolation and identification of MNAR. GST-ERβ-LBD and GST-ERα-LBD were expressed in BL21 (DE3) E. coli (GST is Glutathione S-Transferase). Cell culture in LB at OD_{0.6} was induced with 0.1 mM IPTG, incubated for 3 hr at 25° C. and cells were pelleted by centrifugation. Bacterial cells were sonicated 5 ml of 20 mM Tris pH 7.2, 500 mM NaCl, 1 mM DTT, 0.2 mM EDTA, 0.1 mM PMSF, centrifuged at 40,000 g for 30 min. To immobilize GST-ERβ-LBD and GST-ERα-LBD cell extract was incubated with 50% glutathione agarose slurry in 20 mM Tris pH 7.2, 180 mM NaCl, 1 mM DTT, 0.005% NP40 (Binding Buffer) at 4° C. MCF7 cell extract was prepared using NE-PER Nuclear and Cytoplasmic Extraction Reagents (Perbio Science AB, Pierce, Rockford, Ill.) according to manufacture instructions. 0.5 mg of MCF7 cell extract was incubated at 4° C. with either GST or GST-ER-LBD fusion protein with or without 17-β-estradiol (0.1 μM) at 4° C. Bound material was boiled off the agarose beads in 2× SDS buffer and purified on 10% PAA Gel. The gel was silver stained.

Method 2:

Mass Spectrometric Analysis of Purified Proteins. Protein samples purified by electrophoresis were excised manually, then reduced, alkylated and digested with trypsin (Promega, Madison, Wis.) in situ using an automated in-gel tryptic digestion robot ((Houthaeve T, 1997)). After digestion, peptide extracts were concentrated to a final volume of 10-20 μl using a Savant Speed Vac Concentrator (ThermoQuest, Holdbrook, N.Y.). Peptide extracts were analyzed on an automated microelectrospray reversed phase HPLC. In brief, the microelectrospray interface consisted of a Picofrit fused silica spray needle, 50 cm length by 75 um ID, 8 um orifice diameter (New Objective, Cambridge Mass.) packed with 10 um C18 reversed-phase beads (YMC, Wilmington, N.C.) to a length of 10 cm. The Picofrit needle was mounted in a fiber optic holder (Melles Griot, Irvine, Calif.) held on a home-built base positioned at the front of the mass spectrometer detector. The rear of the column was plumbed through a titanium union to supply an electrical connection for the electrospray interface. The union was connected with a length of fused silica capillary (FSC) tubing to a FAMOS autosampler (LC-Packings, San Francisco, Calif.) that was connected to an HPLC solvent pump (ABI 140C, Perkin-Elmer, Norwalk, Conn.). The HPLC solvent pump delivered a flow of 50 uL/min which was reduced to 250 nL/min using a PEEK microtight splitting tee (Upchurch Scientific, Oak Harbor, Wash.), and then delivered to the autosampler using an FSC transfer line. The LC pump and auto sampler were each controlled using their internal user programs. Samples were inserted into plastic autosampler vials, sealed, and injected using a 5 ul sample loop. Extracted peptides from the surface digests were concentrated 10-fold using a Savant Speed Vac Concentrator (ThermoQuest, Holdbrook, N.Y.), and then were separated by the microelectrospray HPLC system using a 50 minute gradient of 0-50% solvent B (A: 0.1M HoAc, B: 90% MeCN/0.1M HoAc). Peptide analyses were done on a Finnigan LCQ-DECA ion trap mass spectrometer (ThermoQuest, San Jose, Calif.) operating at a spray voltage of 1.5 kV, and using a heated capillary temperature of 125° C. Data were acquired in automated MS/MS mode using the data acquisition software provided with the instrument. The acquisition method included 1 MS scan (375-600 m/z) followed by MS scans of the top 2 most abundant ions in the MS scan. The instrument then did a second MS scan (600-1000 m/z) followed by MS/MS scans of the top 2 most abundant ions in that scan. The dynamic exclusion and isotope exclusion functions were employed to increase the number of peptide ions that were analyzed (settings: 3 amu=exclusion width, 3 min=exclusion duration, 30 secs=pre-exclusion duration, 3 amu=isotope exclusion width). Automated analysis of MS/MS data was performed using the SEQUEST computer algorithm ((Houthaeve T, 1997)) incorporated into the Finnigan Bioworks data analysis package (ThermoQuest, San Jose, Calif.) using the non-redundant (NR) database of proteins obtained from the NCBI genome center.

Method 3:

MNAR Cloning: Three pairs of oligonucleotides were designed and used to clone the N-terminal, middle, and C-terminal portions of MNAR Oligo #1: TAGGATCCAGATGGCGGCAGCCGTTCTGAG (SEQ ID NO 3) and oligo #2: CGATCAGGATCCCAAAGC (SEQ ID NO 4) were used to clone the N-terminal portion. Oligo #3: GCTTTGGGATCCTGATCG (SEQ ID NO 5) and oligo #4: CAAGGAGATCTCCACATC (SEQ ID NO 6) were used to clone the middle portion. Oligo #5: GATGTGGAGATCTCCTTG (SEQ ID NO 7) and oligo #6: GCTAGGAGTCAGGCTCTG (SEQ ID NO 8) were used to clone the C-terminal portion. 1×10⁷ MCF7 cells were lyzed in Trizol (Life Technologies, Rockville, Md.) for total RNA isolation according to protocol. 40 ng of total RNA and 400 nM of oligos was used in a RT-PCR reaction using One Step RT-PCR kit (Qiagen, Valencia, Calif.) to amplify MNAR starting with 30 min at 50° C., followed by 15 min at 95° C., and then 30 cycles of 30 sec at 95° C., 30 sec at 55° C., 60 sec at 72° C., and extension at 72° C. for 7 min. The respective products were cloning into pT-Adv vector (Invitrogen, Carlsbad, Calif.) and sequenced. Full length MNAR was assembled by restriction enzyme digestion and ligation. MNAR was also independently cloned by using oligo #1 and #6 from a human lymphoma marathon cDNA library according to protocol (BD Biosciences Clontech, Palo Alto, Calif.). The MNAR cloned from MCF-7 was identical to the MNAR cloned from marathon cDNA library. A 5′RACE was performed using oligo #7 CCGMGCCAAGACACACAGTGCTGCTGGMTAG (SEQ ID NO 9) and adaptor primer 1 oligo form marathon cDNA kit (BD Biosciences Clontech, Palo Alto, Calif.) to obtain additional sequence information.

Method 4:

MNAR Expression Analysis. Total RNA (10 ug) from HepG2, SaOS2, HOBs 02/02, COS-7, MCF-7. T47D, MDA231, Ishikawa, 1-10, LNCaPLN3, LNCaPFGC, JCA, TSUPRL cells were prepared by Trizol, separated on gel, transferred to membrane, and probed with radiolabeled oligo #7 corresponding to nucleotide 367 to 400 of cloned MNAR. Another oligo probe #8 CTGGAGAAAAAAGGGGCAGAGATAAAGAGT (SEQ ID NO 10), corresponding to nucleotide 1480 to 1509 of deposited Genbank sequence, was used to probe MCF-7 RNA in another Northern.

A second MNAR expression analysis was performed. Radiolabeled oligonucleotide probe corresponding to nucleotide 367 to 393 of MNAR was hybridized to a human multi-tissue northern blot II (BD Biosciences Clontech, Palo Alto, Calif.) in Perfect Hybridization buffer (Sigma-Aldrich, St. Louis, Mo.) at 42° C. The blot was washed three times in 0.2×SSC, 0.1% SDS at 42° C. and exposed to film. β-Actin probe was used as a control.

Method 5:

Transient Transfection Analysis: HepG2 and COS-7 cells were routinely maintained in DMEM supplemented with 10% FBS. Either 50,000 cells per well of HepG2 or 30,000 per well of COS-7 were seeded in 96 well plates in DMEM without phenol red supplemented with 10% charcoal stripped serum. 100 ng of 2×ERE-tk-Luc reporter, 10 ng of pCMV-O-gal internal control, either 1 ng of pcDNA3.1 ERα or 5 ng of pcDNA3.1 ERβ expression vector with or without increasing amount of pcDNA3.1 MNAR or pcDNA3.1 SRC3 expression plasmids were transfected using 0.5 ul of lipofectamine 2000 (Life Technologies, Rockville, Md.) according to protocol. Cells were stimulated either with 10⁻⁸M 17-b-estradiol, tamoxifen, or ICI for 24 hr before being processed for luciferase and β-Galactosidase activity.

Method 6:

MNAR-ER Interaction Analysis:

MNAR-Nuclear receptors interaction analysis was performed. SF9 cells extract, overexpressing full-length flag-MNAR, were incubated with in vitro transcribed/translated [35S]-labeled full-length ERα (FIG. 5A), ERβ (FIG. 5B), androgen (AR) (FIG. 5C) and glucocorticoid receptors (GR) (FIG. 5D) with and without their corresponding ligands, all at 1 μM. Formed complexes were isolated using anti-FLAG sepharose beads, boiled in 2×SDS buffer and loaded on SDS gel. The gel was dried and subjected to autoradiography.

Method 7:

ER-MNAR-cSrc Interaction Analysis. In vitro transcribed/translated ERα with (FIG. 6A, lanes 4, 5 and 8, 9) or without MNAR (FIG. 6A, lanes 2, 3 and 6, 7) in presence (FIG. 6A, lanes 3, 5, 7 and 9) or absence of 1 μM E2 (FIG. 6A, lanes 2, 4, 6 and 8), were incubated with purified cSrc (FIG. 6A, lanes 2-5) or Lck (FIG. 6A, lanes 6-9). Formed complexes were isolate by pulled-down with anti-cSrc or anti-Lck antibodies and protein A agarose, boiled in 2×SDS buffer and resolved on an SDS-PAGE gel. Gel was dried and subjected to autoradiography.

Method 8:

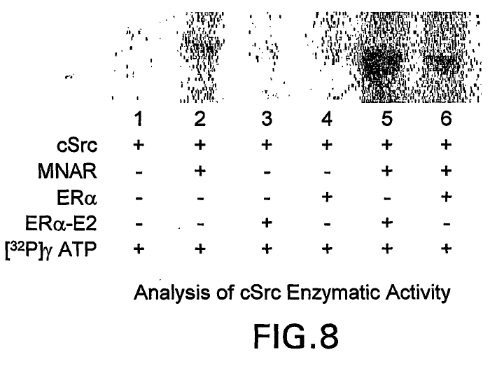

Analysis of cSrc Enzymatic Activity. The cSrc tyrosine kinase activity was evaluated in the absence (FIG. 8, lanes 1 and 2) or presence of ER (FIG. 8, lanes 3-6), 17β-estradiol (FIG. 8, lanes 3 and 5), and MNAR (FIG. 8, lanes 2, 5 and 6) using acidified enolase as a substrate. Before reaction proteins were incubated for 1 hr at 4° C. Phosporylation reaction was started by addition of [32P]-γATP and continued at 30° C. for 10 min. Reaction was stopped by addition of 2×SDS buffer. Phosphorylated proteins were separated by SDS-PAGE and the dried gel was subjected to autoradiography.

Method 9:

MNAR Effect on ER-Mediated Transcription.

A—HepG2 cells were transfected with expression plasmids for ERα, MNAR and/or SRC3. Luciferase gene expression, driven by a 2×ERE-tk-reporter, was evaluated in cells treated with E2, 4(OH)-tamoxifen, or ICI 182,780 all at 10 nM. B—HepG2 cells were transfected with expression vectors for ERαand MNAR. Luciferase gene expressions, driven by a 2xERE-tk-reporter, were evaluated in cells treated with 10 nM E2, 10 nM E2 plus 1 μM PP2, or 1 μM PD98059. C—MCF-7 cells were transfected with 100 nM antisense (AS) CATGGAGATGTCCCGGAACAGTGCA (SEQ ID NO: 11) from Sequitur, Inc. (Natick, Mass.) or reverse control (C) oligomers complexed with 1 μg/ml of Lipofectamine 2000 in white media. After 24 hours cells were stimulated with 10 nM E2, 24 hours later cells were lysed in guanidium buffer and total RNA was isolated from lysates. TaqMan analysis was performed using a primer/probe set designed to target MNAR, pS2 and Cathepsin D. D—Extracts of MCF7 cells transfected with 100 nM antisense (AS)—1, or reverse control (C)—2 oligomers stimulated with 10 nM E2 were used for Western Blotting Analysis with rabbit polyclonal MNAR antiserum. MNAR antiserum was generated against 11-mer peptide encoding amino acids 509-520 (SHRKGDSNANSD) (SEQ ID NO: 31) of the MNAR.

Method 10:

MNAR and ER Interaction and Activation of Src Kinase

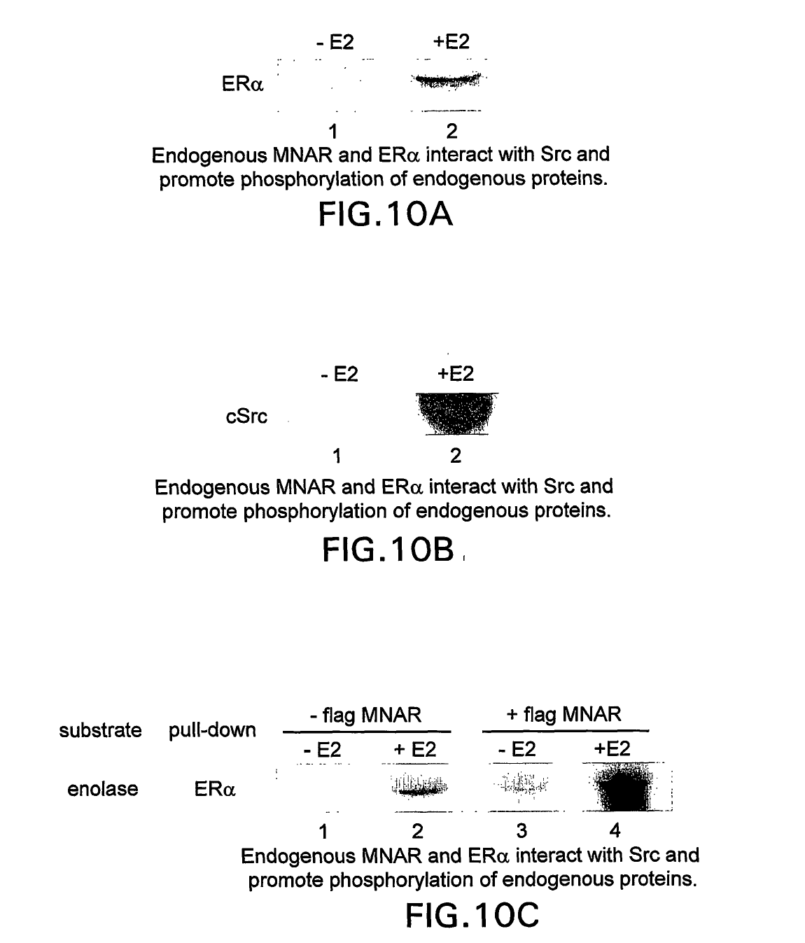

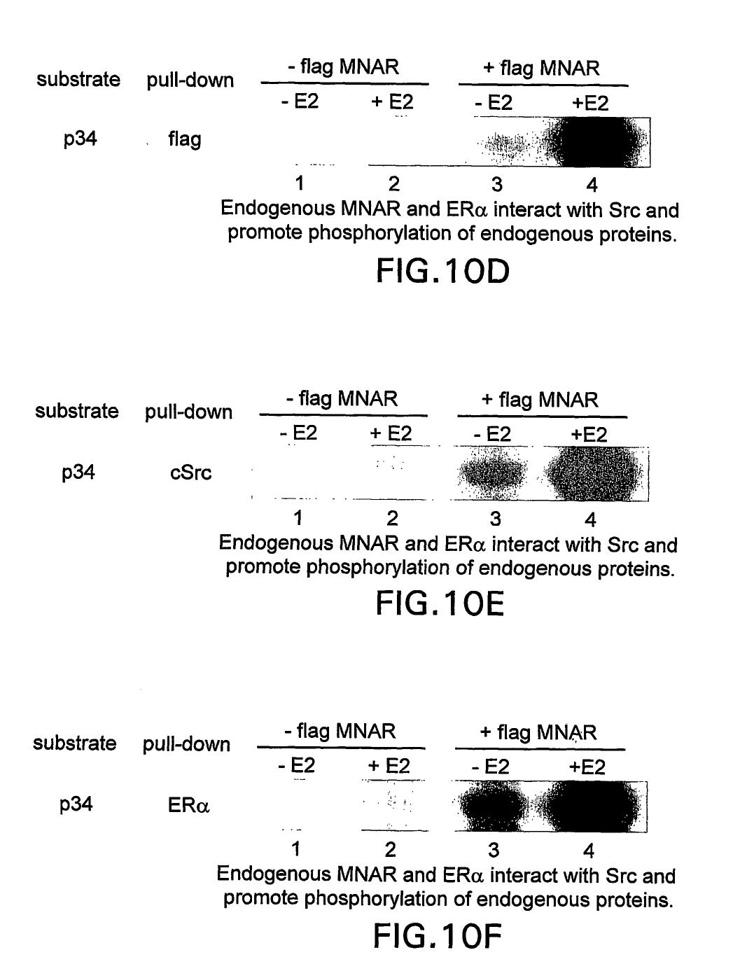

MNAR and ERα interact with Src and promote phosphorylation of endogenous proteins. MCF-7 cells transfected or not transfected with FLAG-MNAR expression vector were not treated (FIG. 10, lanes 1 and 3) or treated with E2 at 10 nM for 5 minutes (lanes 2 and 4). FLAG-MNAR (panel A), cSrc (panel B), or ERα (panels C, D and E) were immunoprecipitated from the cell extracts using corresponding antibodies. Immunoprecipitates were incubated with [32P]γATP for 30 minutes at 30° C. (panels A-C), or with [32P]-γATP and enolase (panel D). Kinase reaction was stopped by boiling in 2×SDS buffer. Phosphorylated material was loaded on SDS gel, which was then dried and subjected to autoradiography. Material obtained by ERα pull down from cells not transfected with FLAG-MNAR, untreated (1) and treated with E2 (2) was probed with MNAR-antiserum (panel E).

Method 11:

MNAR affect on E2 Induced Erk Kinase 1 and 2 Activation

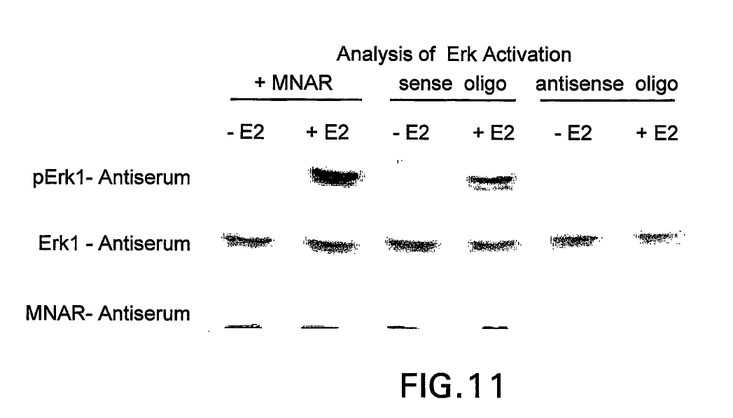

It has been previously reported that activation of Src by estradiol triggers Ras/Erkₛ kinase pathway. We expected that MNAR induced Src activation may lead to activation of Erk kinases 1 and 2. We evaluated the level of Erk activation by E2 in MCF7 cells, cells overexpressing MNAR, and cells in which MNAR expression was substantially attenuated by treatment with MNAR antisense oligonucleotide (SEQ ID NO:11). Antibodies that recognize phosphorylated Erk 1 and 2 were used to detect E2 induced stimulation of Erk 1 and 2 phosphorylation. Extracts of MCF7 cells (FIG. 11, lanes 3 and 4) transfected with MNAR expression plasmid (FIG. 11, lanes 1 and 2), or MNAR antisense oligonucleotide at 100 nM (FIG. 11, lanes 5 and 6); unstimulated (FIG. 11, lanes 1, 3 and 5), or stimulated for 5 minutes (FIG. 11, lanes 2, 4 and 6) with 10 nM E2 were used for Western blotting analysis with antibodies against phosphorylated Erk 1 and 2—panel A; Erk_{1/2} antibodies—panel B; MNAR antibodies—panel C. MNAR antisense oligonucleotide (SEQ ID NO:11) was developed at Sequitur Inc. (Natick, Mass.).

Method 12:

MNAR Structural-Functional Analysis

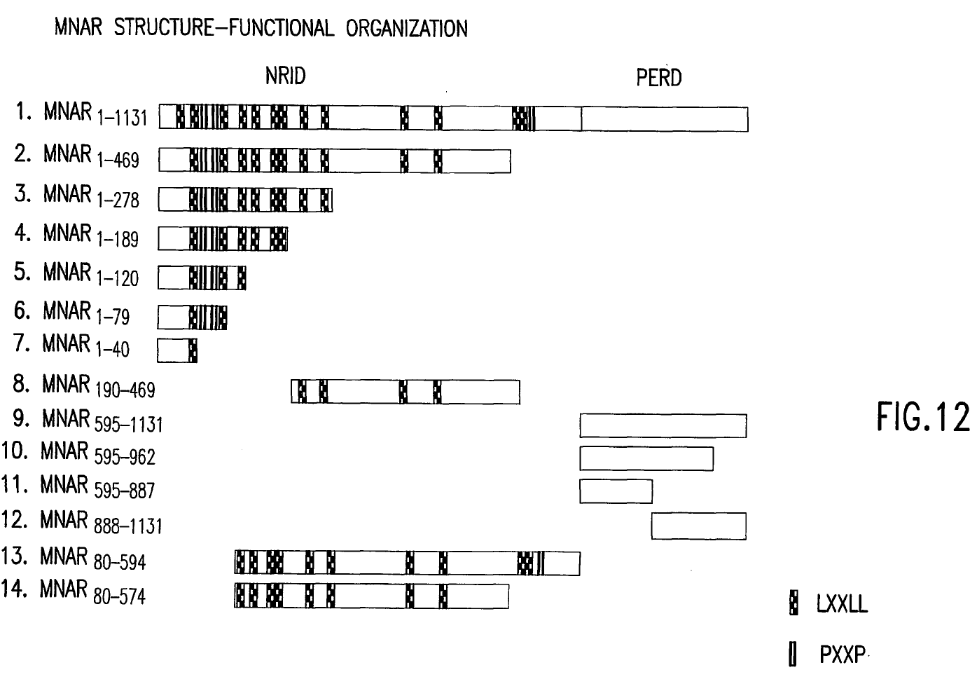

MNAR sequence analysis revealed that the N-terminal portion of its molecule contains multiple LXXLL motifs previously found to be important for interaction with nuclear hormone receptors, while at the C-terminus, MNAR contains a region rich in both proline and glutamic acid. To delineate the regions of MNAR that are responsible for interaction with estrogen receptor and c-Src tyrosine kinase, we performed functional evaluation of MNAR deletion mutants (FIG. 12). A series of C-terminal truncations of MNAR was generated using specially designed oligonucleotides, by amplifying appropriate fragments and cloning them into appropriate expression vectors. These mutants were tested for their abilities to enhance ER-stimulated transcription when transfected into HepG2 cells.

Method 13:

Screening for Compounds that Affect Kinase Activity.

Compounds are screened as follows. The reaction buffer used is 50 mM Tris-HCl, pH 7.0, 50 mM MgCl₂, 50 uM Na₃VO₄. The substrate peptide is obtained and 6 mg dissolve in 1.7 mL of RB and store at −20° C. ATP to be used is at 400 uM final concentration. An inhibitor is to be used, for example, Piceatannol—3 mM (stored at 4° C. and protected from light).

First, ER incubation with E2. For example, 10 μl of commercially available ER (Panvera) is incubated with 40 μl of RB and 0.5 μl of E2 at 10⁻⁶M. The mix is incubated at RT for 1 hour.

Next, the mixture is placed in the presence or absence of MNAR, E2, and ER. The solutions are incubated at RT for 30 minutes.

To each tube, 10 μl ATP (final concentration 400 μM) is added along with 10 μl substrate peptide. This is incubated for 60 minutes at 30° C.

The reaction is stopped by adding 10 μl of 3 mM piceatannol. Now the solution is incubated at RT for 5 minutes.

Dilution and peptide immobilization is performed. 10 μl Reaction is diluted with 20 ml H₂O. 200 μl of dilution is added to wells of NeutrAvidin Plate Washing is performed by adding 200 μl of PBS 5× for 3 minutes each. This is followed by addition of Anti-P-Tyr antibody. The antibody is diluted in PBS and BSA and the solution is covered and allowed to incubate at 37° C. for one hour. The mixture is washed the same as above.

Then a substrate reaction is generated by adding 100 μl of Pierce substrate and reading in Victor using the HRP protocol.

Experimental Analysis and Results

### Example 1

MNAR Isolation and Identification, Cloning of cDNA Encoding MNAR.

To better understand the tissue-selective action of ER ligands, we established a proteomics approach to evaluate the expression and activity of ER-interacting proteins in different cell lines. We identified MNAR using GST-pull down approach. A GST-ERβ ligand-binding domain (LBD) was incubated with a MCF-7 cell extract. FIG. 2A presents a silver-stained gel of the fractions obtained by the pull-down experiment. A number of proteins were detected that specifically interacted with GST-ERβ-E2, but not with GST-ERβ, or GST alone.

Corresponding bands were excised, digested with trypsin, concentrated, and separated by the microelectrospray HPLC system. Peptide analysis was done on a Finnigan LCQ-DECA ion trap mass spectrometer (ThermoQuest, San Jose, Calif.). Automated analysis of mass spectrometry data was performed using the SEQUEST computer algorithm ([Houthaeve T, 1997]) incorporated into the Finnigan Bioworks data analysis package (ThermoQuest, San Jose, Calif.) using the non-redundant database of proteins obtained from the NCBI genome center.

FIG. 2A shows a silver-stained gel resulting from the experimental steps as outlined above in Method 1. Cell extract was incubated with Glutathione beads—1, bacterially expressed GST-ERβ-LBD fusion protein in the absence of ligand—2, or presence of E2—3, or 4(OH) tamoxifen—4, both at 10⁻⁷M. ER-interacting proteins were isolated using anti-GST agarose and separated on SDS-PAGE. The gel area containing the corresponding band was excised and subjected to trypsin digestion.

The bands corresponding to the proteins revealing ligand-dependent interaction were excised, digested with trypsin and identified using mass spectrometry based peptide microsequencing. Together with known and well-characterized coactivators, such as SRC3 and DRIP205, we identified a protein that had not been previously shown to interact with ERs. This protein interacted with the GST-ERβ-LBD in the presence of E2 and had an apparent molecular weight of approximately 120 kDa (FIG. 2A).

Peptide sequence analysis revealed that this protein could be related to a proline and glutamic acid rich protein—-p160 (Genbank U88153), previously isolated by pull-down with the Src homology domain 2 (SH2) of p56^{lck} (Lck) (21). FIG. 2B presents sequences of the peptides identified by the mass spectrometry and their position in the MNAR molecule.

Searching the NCI database we also identified several ESTs that matched this protein. One of them, AL03939, matched at the N-terminus and extended the 5′ sequence an additional 100 bp. The aligned sequence was used to design oligonucleotide primers to clone this protein from MCF-7 cells (22).

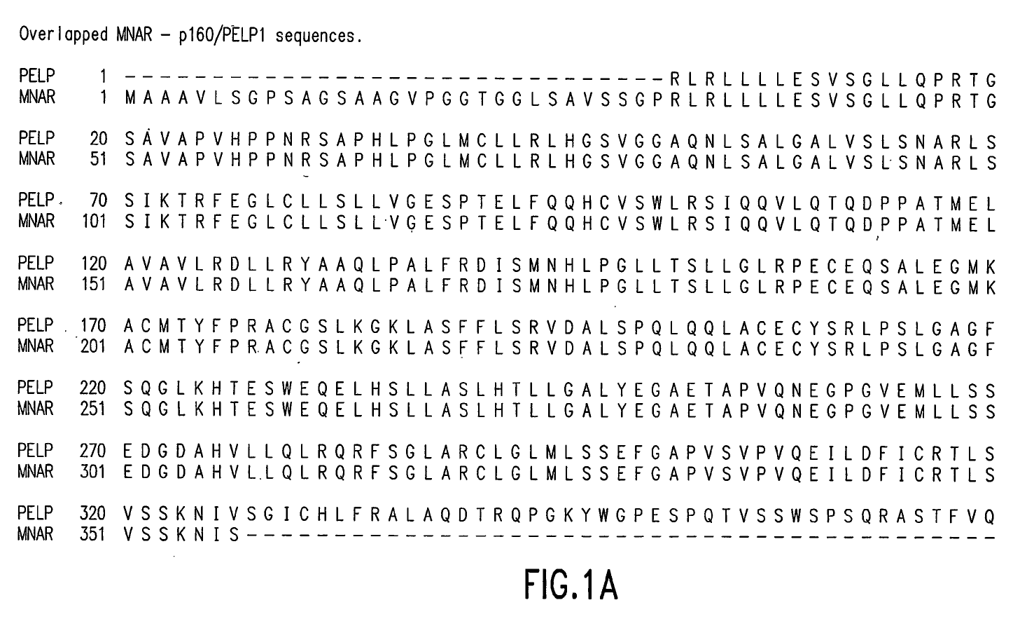

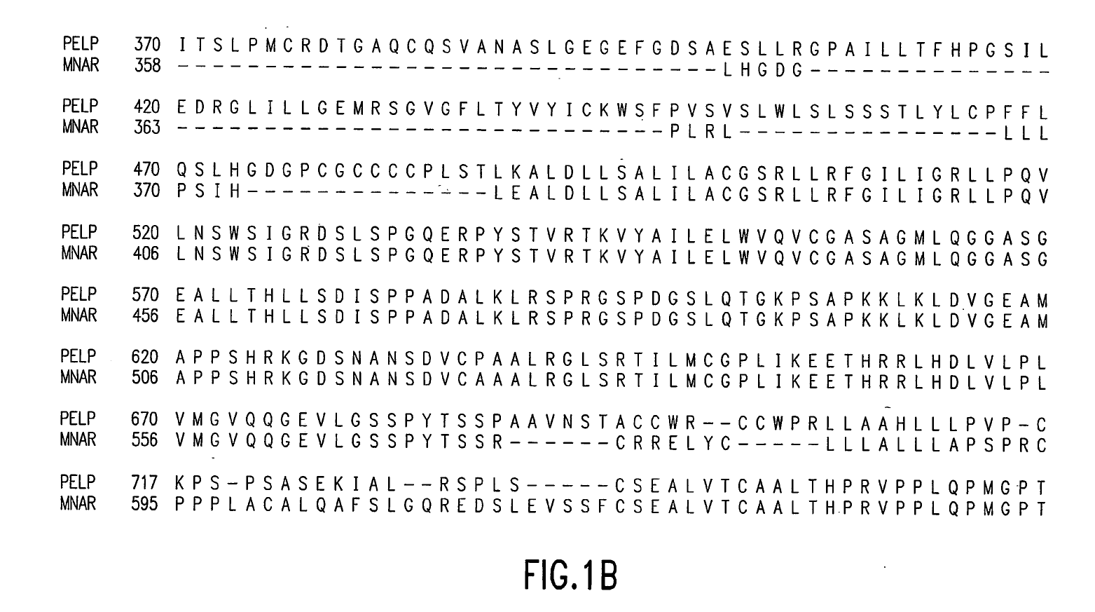

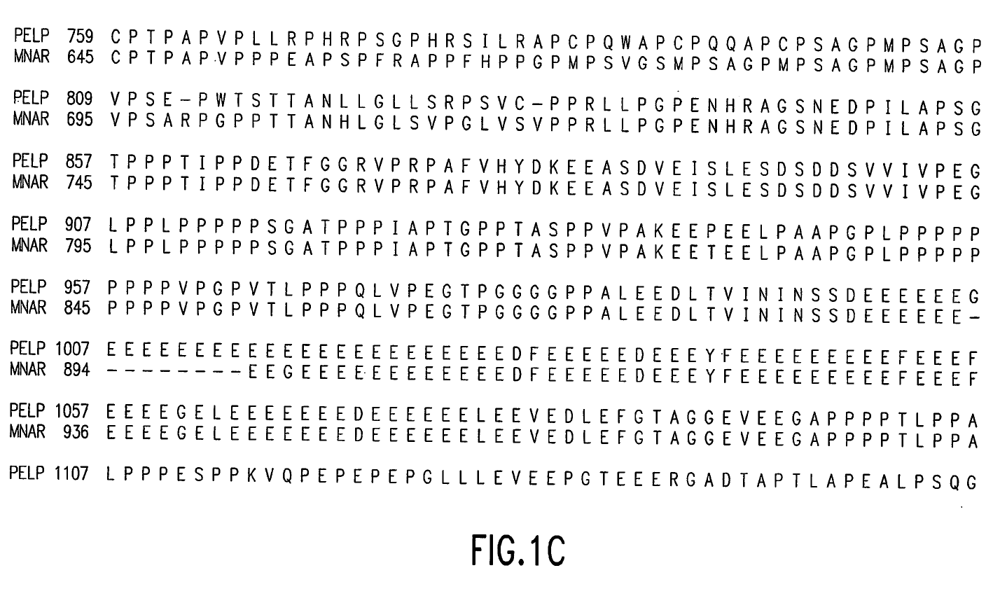

However, the sequence of the cloned protein (see FIG. 1) differed substantially from the p160 (Genbank U88153). To differentiate between the two sequences, and to avoid confusion with the family of nuclear receptor coactivators that also are referred as p160s ((Voegel et al., 1998), (Hong et al., 1997), (Anzick et al., 1997), [Suen, 1998]), we named the protein that we cloned from MCF7 cells MNAR.

Sequence alignment of MNAR and p160 (FIG. 1A) indicated that MNAR does not contain nucleotides from 1075 to 1510 and 3125 to 3151 that are present in the p160 (Genbank U88153). In addition, there were 10 single base pair gaps and one double base pair gap compared to the p160 sequence.

MNAR was also independently cloned from a human lymphoma Marathon cDNA library (Invitrogen). Importantly, the MNAR cloned from MCF-7 cells was identical to the MNAR cloned using marathon cDNA library. To obtain additional sequence information, we performed the 5′RACE with primers from marathon cDNA kit (Clonetech). Using 5′RACE, stop codons were found in all three reading frames 5′ of the putative start codon of MNAR. Conceptual translation of the MNAR clone resulted in an 1130 amino acid protein with a calculated molecular weight of 119.6 kDa. This clone contained all peptide sequences initially identified by mass spectrometric analysis (FIG. 2B).

Sequence analysis has revealed multiple LXXLL motifs localized in the N-terminal portion of MNAR molecule. We putatively call it nuclear receptor interaction domain (NRID). Similar motifs in other transcription factors have been shown to interact with a hydrophobic groove on the surface of the ligand-binding domain of nuclear hormone receptors (Heery et al., 1997), (Torchia et al., 1997) (23). Interesting features of the MNAR molecule are extended proline (PRD) and glutamic acid (ERD) rich domains localized in the C-terminal part of the MNAR molecule. No homology to these domains has been found in the NCl database.

### Example 2

MNAR Expression Analysis. MNAR expression in HepG2, SaOS2, HOBs 02/02, COS-7, MCF-7. T47D, MDA231, Ishikawa, 1-10, LNCaPLN3, LNCaPFGC, JCA, TSUPRL cell lines was evaluated using Northern blotting analysis. For this purpose we used an oligo-probe derived from the N-terminal portion of MNAR, which is common between MNAR and the deposited p160 sequence (Genbank U88153). Expression of MNAR mRNA was detected in almost all cell lines (FIG. 3), as estimated by the presence of a 4 kb band (FIG. 3). The level of the MNAR expression varied significantly among different cell lines. It was high in liver (HepG2) and prostate (LNCaPLN3, LNCaPFGC, JCA, TSUPRL) cells, and low to undetectable in breast tumor cells that do not express ER (T47D, MDA231) (FIG. 3). To evaluate whether p160 is expressed in MCF-7 cells we used Northern blotting analysis with oligo-probe to the region of p160 that is missing in MNAR sequence and is encoded by nucleotides 1480 to 1509 of deposited to Genbank p160 sequence. Using this probe we failed to detect any message (data not shown). This result indicates that the major form of this protein, expressed in MCF-7 cells, matches well with the MNAR, while the mRNA encoding the p160 is not expressed or expressed at very low levels.

MNAR distribution in different tissues was analyzed by Northern blotting. Again, we used an oligo-probe derived from the N-terminal portion of MNAR, which is common between MNAR and the deposited p160 sequence (Genbank U88153). Once again, expression of MNAR mRNA was detected in almost all tissues (FIG. 4), as estimated by the presence of a 4 kb band (FIG. 4). The level of MNAR expression appears to vary among different tissues.

### Example 3

MNAR Specifically Interacts with Estrogen Receptors

With full-length flag-MNAR, we further evaluated whether MNAR interaction with estrogen receptors is affected by ER ligands. Full-length flag-MNAR, overexpressed in SF9 cells using baculoviral expression system, was used in a pull-down experiment with in vitro transcribed/translated full-length unliganded ER α and β, or receptors liganded with E2, or 4-OH tamoxifen. Formed complexes were isolated using anti-FLAG sepharose beads.

FIGS. 5A and 5B reveals that ER α and β interact with MNAR, and that this interaction is enhanced by E2 and inhibited by 4(OH) tamoxifen. We also evaluated MNAR interaction with some other nuclear hormone receptors. Full-length flag-MNAR was incubated with transcribed/translated androgen (AR) and glucocorticoid receptors (GR) with and without their corresponding hormone. The data indicated that AR and GR ligand dependently interacted with MNAR (FIGS. 5B and 5C). This interaction may explicate the nongenomic activity of these receptor ligands. Detailed investigation of these and other nuclear hormone receptor's crosstalk with MNAR and the Src phosphorylation cascade is an important subject for future investigation.

### Example 4

MNAR interaction with c-Src and Lck is enhanced by E2-liganded estrogen receptors. Considering that a protein homologous to MNAR, p160, was initially identified via pull-down with the SH2 domain of p56^{lck} (Lck), we next examined whether MNAR interacts with members of Src family tyrosine kinases and affects their interaction with ER.

To address this question, we have used in vitro transcribed/translated estrogen receptors ERα, ERβ and MNAR. ER with and without 17β-estradiol (E2), alone or together with MNAR were incubated with commercially available, partially purified cSrc or Lck (Upstate Biotechnology). Formed complexes were pulled-down with anti-cSrc or anti-Lck antibodies and protein A sepharose and then resolved on an SDS gel.

The data indicate that ER a interacts with cSrc and Lck (FIG. 6A, lanes 2 and 3, 6 and 7). Surprisingly, this interaction was inhibited by E2. When ERa was incubated with both MNAR and cSrc (FIG. 6A, lanes 4 and 5) or Lck (FIG. 6A, lanes 8 and 9), significant stimulation of Src-ER-MNAR complex formation was detected in the presence of E2 (FIG. 6A, lanes 5 and 9) compared to unliganded ERa (FIG. 6A, lanes 4 and 8). Since MNAR itself does not interact well with cSrc or Lck (FIG. 6A, lane 11), and interaction of cSrc and/or Lck with ERs is inhibited by E2 (FIG. 6A, lane 3,), we conclude that all three proteins strongly interact in the presence of E2 and that MNAR is the key to ER-Src interaction. Identical data were obtained for ERβ (FIG. 6B).

Previous studies from Auricchio and colleagues suggested that ER directly binds to the SH2 domain of Src and that this binding is enhanced by E2. A potential explanation for the apparent inconsistency between our data and theirs may rely on the fact that to evaluate ER-Src interaction they used Src pull-downs from cell extracts. MNAR was most likely present in these extracts and would have mediated ER-Src complex formation.

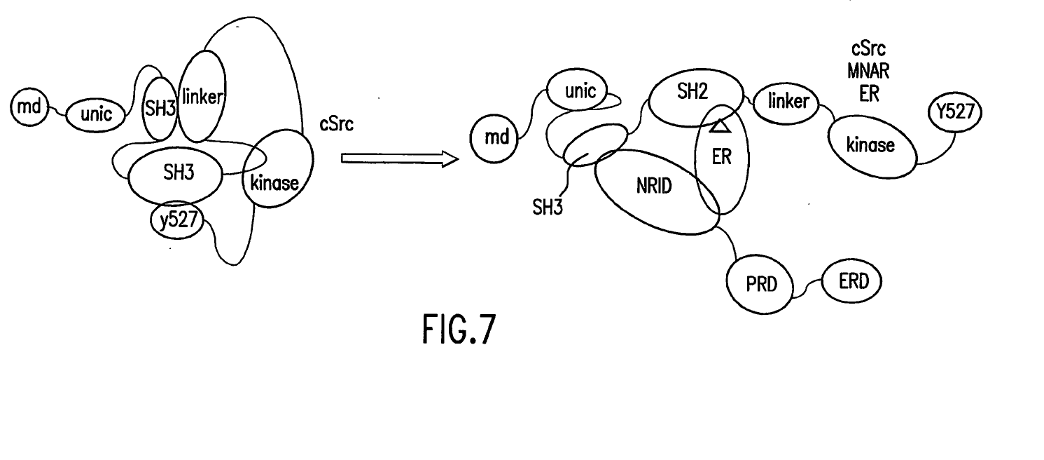

Under basal conditions, the catalytic domain of Src is constrained in an inactive state through intramolecular interactions. Binding of the SH2 domain to the C-terminal phosphorylated tyrosine and SH3 domain to proline-rich region in Src linker domain locks the molecule in an inhibited conformation. (FIG. 7). Full catalytic activation requires release of these restraints.

The kinase activity of Src can be enhanced by binding of phosphotyrosine-containing sequences to the SH2 domain and binding of proline-rich sequences to the SH3 domain. Considering that MNAR contains an extended proline-rich domain (PRD) and the ligand-binding domain of ER contains a tyrosine residue that can be phosphorylated, we hypothesized that these interactions may lead to Src activation (see FIG. 7).

### Example 5

MNAR Stimulates ER-Driven Transactivation. Activation of cSrc in response to EGF has been previously shown to promote ER phosphorylation and to promote phosphorylation of other transcription factors by MAP kinase, thereby stimulating ER-mediated transcription ([Kato, 1996]; [Bunone, 1996]). To evaluate whether MNAR affects transcriptional activity of ER, COS7 cells were cotransfected with ERα (FIG. 6A) or ERβ (FIG. 6B) and MNAR. Luciferase gene expression driven by 2×ERE-tk-reporter was evaluated in cells treated and untreated with 17β-estradiol. Our data indicate that increased MNAR expression correlates with stimulation of ERα and ERβ transcriptional activity (FIGS. 6A and 6B). Next we evaluated whether cSrc inhibitors affect MNAR-promoted ER activation. HepG2 cells transfected with ERα and MNAR (FIG. 8) were treated for 24 hr with 17β-estradiol alone, with 17β-estradiol and selective inhibitor of Src—PP2 (Calbiochem) or with genistein. As in the previous experiment (FIG. 8) MNAR stimulated E2 induced ERα activity. At the same time Src inhibitors PP2 and genesteine inhibited MNAR stimulation (FIG. 8). These data indicate that MNAR induced stimulation of ER activity is linked to the activation of a phosphorylation cascade.

### Example 6

MNAR Overexpression affects Transcriptional Activity of ER

To evaluate if MNAR overexpression affects transcriptional activity of ER, HepG2 cells were transiently cotransfected with plasmids for expression of ERα and MNAR. Luciferase gene expression controlled by a 2×ERE-tk-reporter was evaluated in cells treated with E2, 4(OH)-tamoxifen, or ICI 182,780 all at 10 nM.

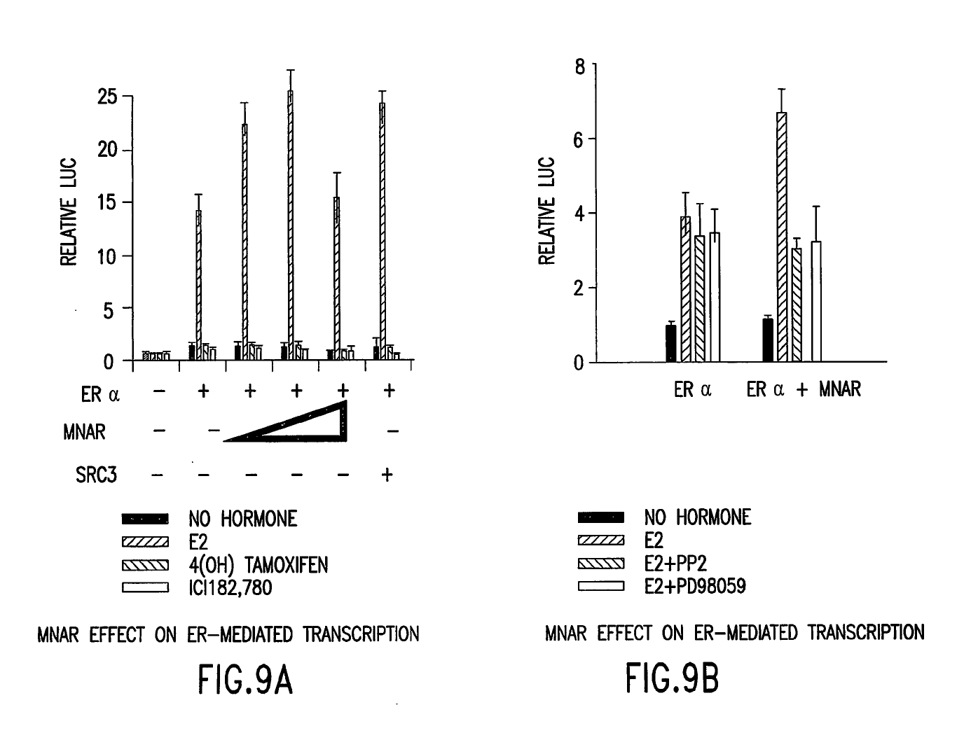

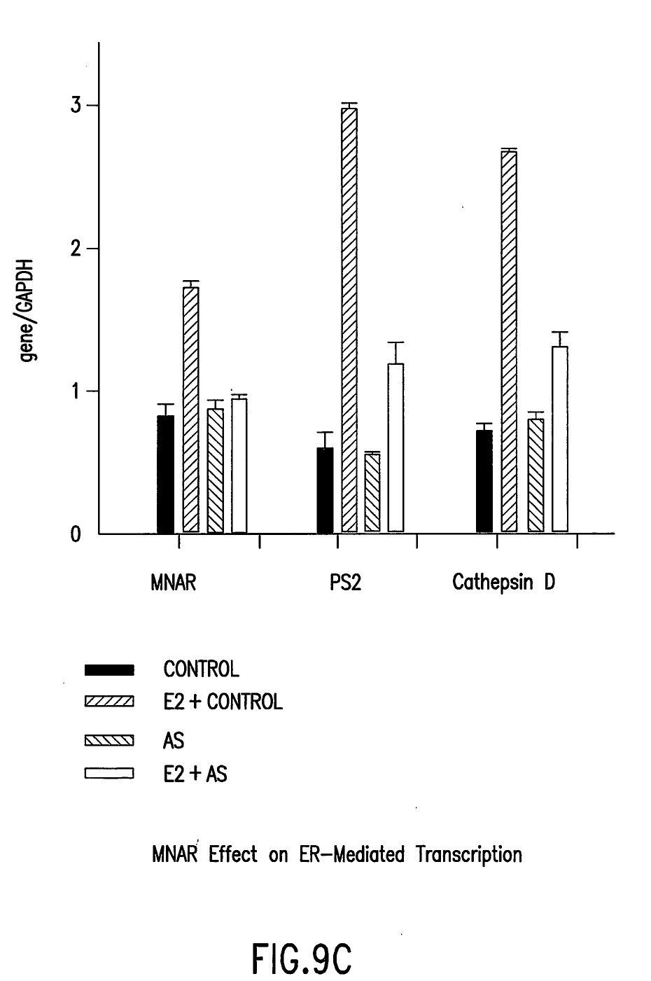

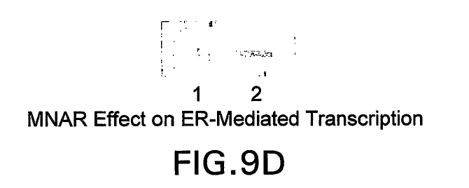

The results (FIG. 9A) indicate that increasing MNAR expression correlates well with stimulation of ERα transcriptional activity. Similar stimulation of ER transcriptional activity was observed with overexpression of the ER-coactivator, SRC3. Our data also indicate that 4(OH)-tamoxifen and ICI 182,780 do not support MNAR stimulation of ER activity. Similar results were observed with ERβ (data not shown).

To investigate if cSrc activation is responsible for the MNAR stimulation of ER activity, HepG2 cells transfected with ERα, MNAR and 2×ERE-tk-reporter plasmid (FIG. 9B) were treated with E2 alone, or E2 and either the cSrc inhibitor, PP2, or the MEK kinase inhibitor, PD98059. We found that both PP2 and PD98059 abrogate MNAR stimulation of ER activity. These results indicate that MNAR promoted stimulation of ER transcriptional activity is linked to the activation of the MAP kinase phosphorylation cascade. We hypothesize that ER phosphorylation by Erk, downstream from Src and MEK kinases, may be responsible for enhancement of ER transcriptional activity, as previously described. In addition, it is possible the that activation of the Src/MAP kinase pathway leads to phosphorylation of some other transcription factors, that are important for ER transcriptional activity. Importantly, these data also suggest that the so-called “nongenomic” action of nuclear hormone receptors through activation of the phosporylation cascade may regulate activity of transcription factors, and, by doing so, ultimately influence gene expression. We speculate that this mechanism may institute the crosstalk between nuclear receptors that are able to interact with MNAR and other transcription factors whose activity is regulated by phosphorylation.

### Example 7

MNAR Affects ER-Mediated Gene Expression

We next used an antisense approach to assess MNAR's role in estrogen regulation of gene transcription. Antisense oligonucleotides (SEQ ID NO:11) were developed (Sequitur Inc., Natick, Mass.) that significantly inhibited MNAR expression in MCF-7 cells, as estimated by TaqMan (FIG. 9C) and Western Blotting (FIG. 9D) analyses. MCF-7 cells were transfected with 100 nM antisense or reverse control oligomers. After 24 hours, cells were stimulated with 10 nM E2, and 24 hours later cells were lysed and total RNA was isolated. TaqMan analysis was performed using a primer/probe set designed to target MNAR, pS2 and Cathepsin D—two genes known to be regulated by estrogens in MCF-7 cells. FIG. 9C presents the levels of MNAR, pS2 and Cathepsin D mRNA expression, normalized to GAPDH mRNA. Interestingly expression of MNAR mRNA was substantially stimulated by E2 treatment. Our data indicate that reduction in MNAR expression lead to dramatic reduction of E2-stimulated pS2 and Cathepsin D expression. Importantly, the MNAR antisense oligonucleotides did not significantly affect the basal level of these genes expression. We expect that ER phosphorylation is probably important for E2-mediated expression of pS2 and Cathepsin D. These data support our position that MNAR is essential for ER regulation of gene expression.

### Example 8

MNAR and ER Interaction and Activation of Src Kinase in MCF-7 Cells

We evaluated whether MNAR and ER interacted and activated Src kinase in MCF-7 cells. MCF-7 cells transfected with a Flag-MNAR expression vector were treated with E2 at 10 nM for 5 minutes. Flag-MNAR (FIG. 10, panel A), ERα (FIG. 10, panel C and D), and csrc (FIG. 10, panel B) were immunoprecipitated from the cell extracts using their corresponding antibodies. Material precipitated using ERα antiserum was probed with rabbit polyclonal anti-MNAR antibody (FIG. 10, panel E). E2-enhancement of ERα-MNAR interaction was clearly detected. Considering that MCF7 cells not transfected with FLAG-MNAR were used for this experiment, this data confirm that endogenous MNAR and ERα interact in MCF7 cells. Immunoprecipitates were incubated with [32P]γATP at 30° C. for 30 minutes. Reaction was stopped by boiling in 2×SDS buffer and resolved on a SDS PMG. Strong MNAR- and E2-dependent phosphorylation of several endogenous proteins was detected. Especially evident was phosphorylation of an endogenous protein with an apparent molecular weight of approximately 34 kDa that coprecipitated with cSrc, ER, and MNAR (see FIG. 10).

Importantly phosphorylation of this protein was detected in material coprecipitated with anti-Src and anti-ER antibodies from cells untransfected with MNAR (FIG. 10, lanes 1 and 2). The phosphorylation was stimulated in presence of E2 (FIG. 10, lane 2) and then dramatically augmented in cells transfected with MNAR (FIG. 10, lanes 3 and 4). These data strongly support our position that MNAR, ER, and cSrc interact in MCF-7 cells, and that the MNAR-ER complex strongly stimulates cSrc kinase activity, promoting phosphorylation of some endogenous protein in MCF-7 cells. An identical phosphorylation pattern was detected when enolase was used as Src substrate (FIG. 10, panel D).

### Example 9

MNAR Affects E2 Induced Erk Kinase 1 and 2 Activation

We next evaluated the level of Erk activation by E2 in MCF7 cells (see FIG. 11, lanes 3 and 4) cells overexpressing MNAR (FIG. 11, lanes 1 and 2) and cells in which MNAR expression was substantially attenuated by treatment with MNAR antisense oligonucleotide (FIG. 11, lanes 5 and 6). Strong E2 induced stimulation of Erk 1 and 2 phosphorylation was clearly detected using antibodies that recognize phosphorylated Erk 1 and 2 (FIG. 11, panel A). This activation was significantly enhanced in cells overexpressing MNAR (FIG. 11, panel A, lanes 1 and 2) and attenuated in cells treated with MNAR antisense oligonucleotides (FIG. 11, panel A, lanes 5 and 6). Neither treatment with MNAR antisense oligonucleotides nor short treatment with E2 affected the level of total Erk 1 and 2 (FIG. 11, panel B). Western blotting analysis indicated that MNAR level was strongly augmented in cells transfected with MNAR expression plasmid (FIG. 11, panel C, lanes 1 and 2) and attenuated in cells treated with MNAR antisense oligonucleotides (FIG. 11, panel C, lanes 5 and 6). These data indicate that MNAR controls E2 induced stimulation of cSrc and Erk 1 and 2 kinases.

### Example 10

MNAR Structural-Functional Organization

MNAR sequence analysis revealed that the N-terminal portion of its molecule contains multiple LXXLL motifs found to be important for interaction with nuclear hormone receptors. At the C-terminus, MNAR contains a region rich in both proline and glutamic acid. To delineate the regions on MNAR responsible for interaction with estrogen receptors and c-src tyrosine kinase, a functional evaluation of MNAR deletion mutants was performed (FIG. 12). A series of C-terminal truncations of MNAR was generated using specially designed oligonucleotides, by amplifying appropriate fragments and cloning them into appropriate expression vectors. These mutants were tested for their abilities to enhance ER-stimulated transcription when transfected into HepG2 cells.

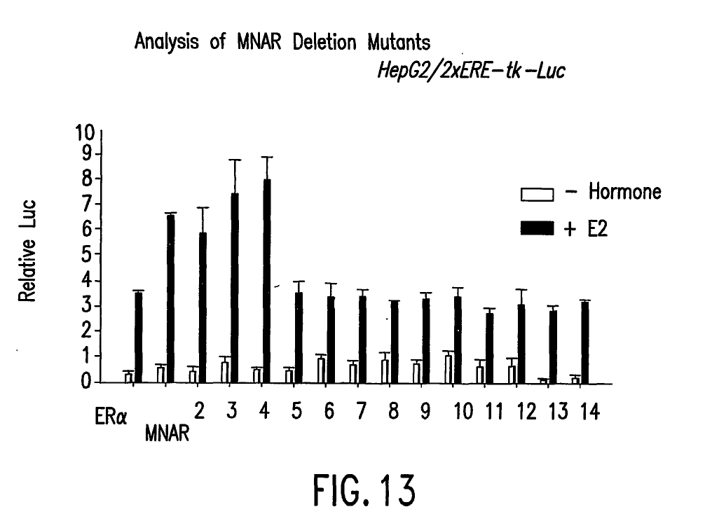

While MNAR mutants containing aa 1-469 (mutant 2, SEQ ID NO: 14), 1-278 (mutant 3, SEQ ID NO:15), and 1-189 (mutant 4, SEQ ID NO:16) are still able to augment ER transcriptional activity. Mutants containing the C-terminal portion of MNAR molecule aa 595-1131 (mutant 9, SEQ ID NO: 17), 595-962 (mutant 10, SEQ ID NO: 18), and 595-887 (mutant 11, SEQ ID NO: 19)) were inactive. These data suggest that the domain that is necessary and sufficient to stimulate ER transcriptional activity is localized to the N-terminal part of NRID between aa 1 and 189 (FIG. 13). This region contains the LXXLL motifs—4^{th} (aa154-159) (SEQ ID NO: 20), 5^{th} (aa 176-181) (SEQ ID NO: 21), and 6^{th} (aa 181-185) (SEQ ID NO: 22) (FIG. 12). To evaluate which of these motifs is responsible for interacting with estrogen receptor, we generated mutants of MNAR with these three LXXLL motifs mutated individually or in combination.

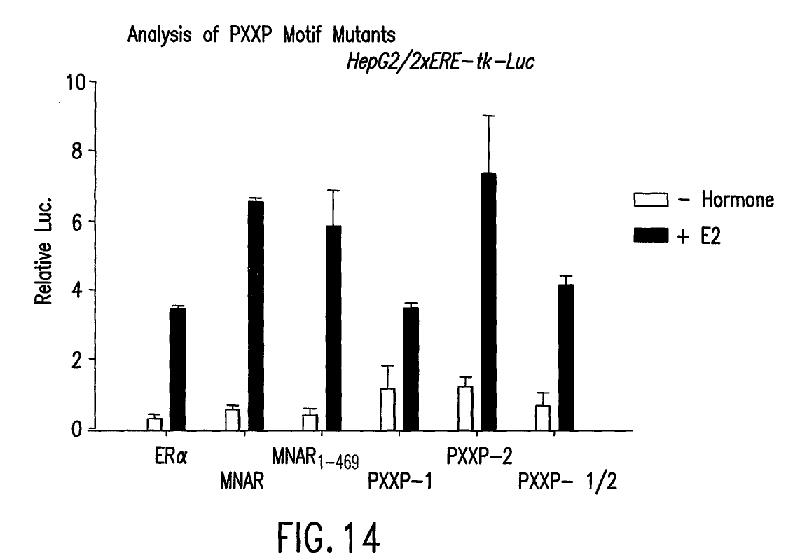

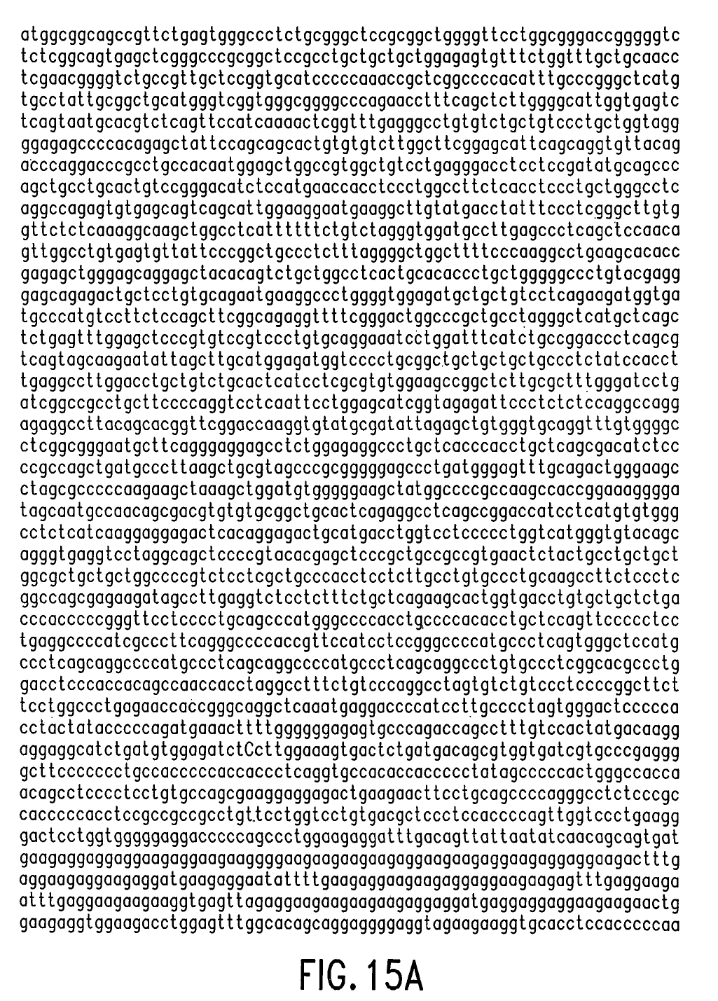

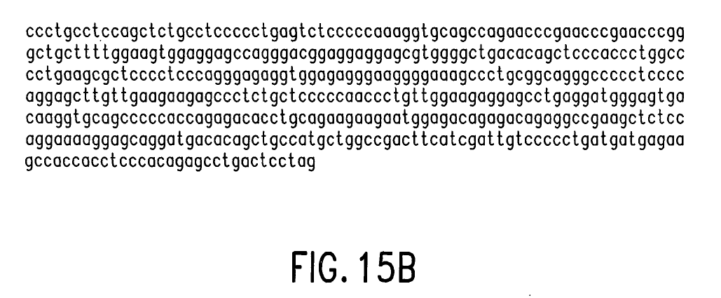

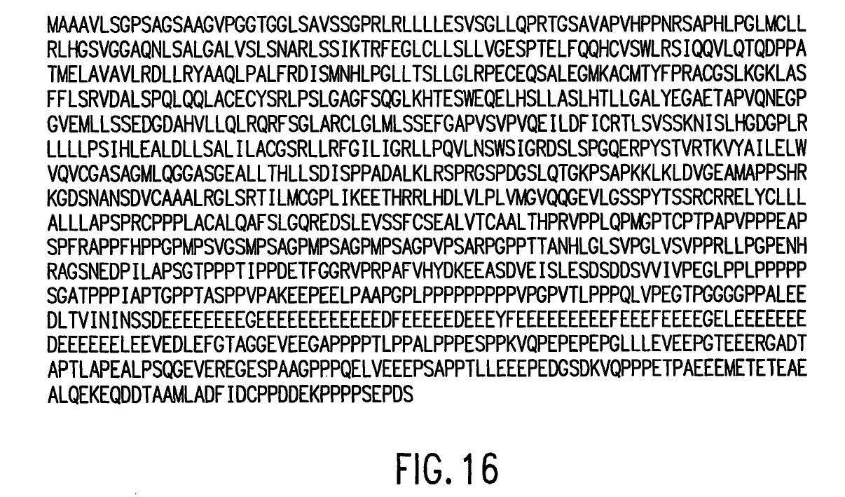

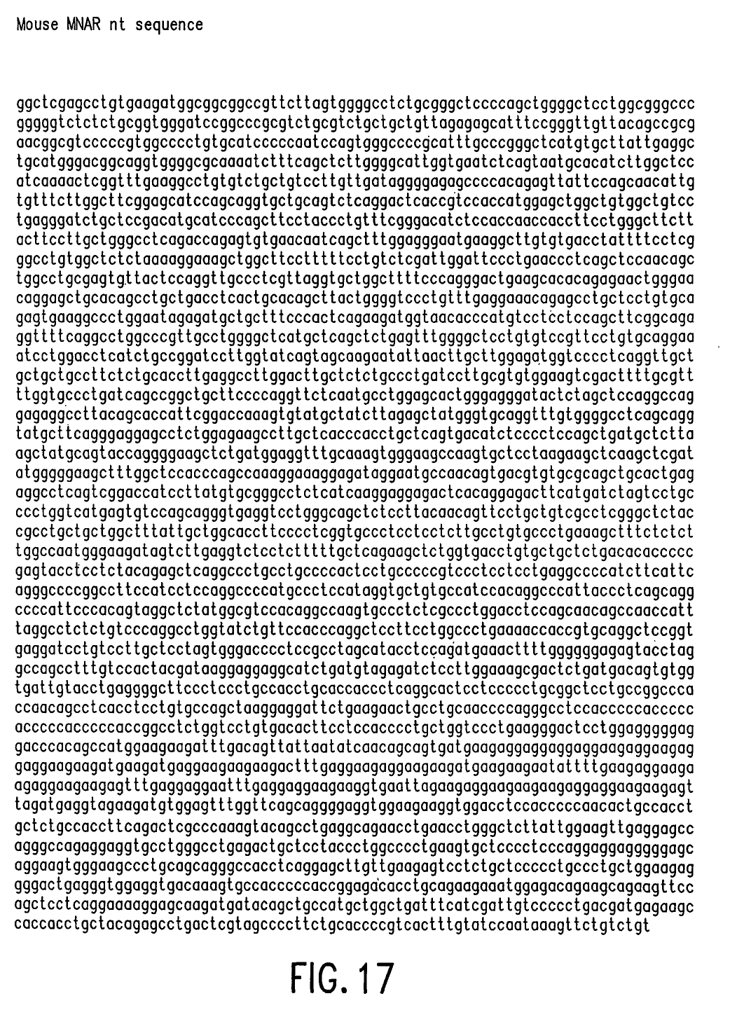

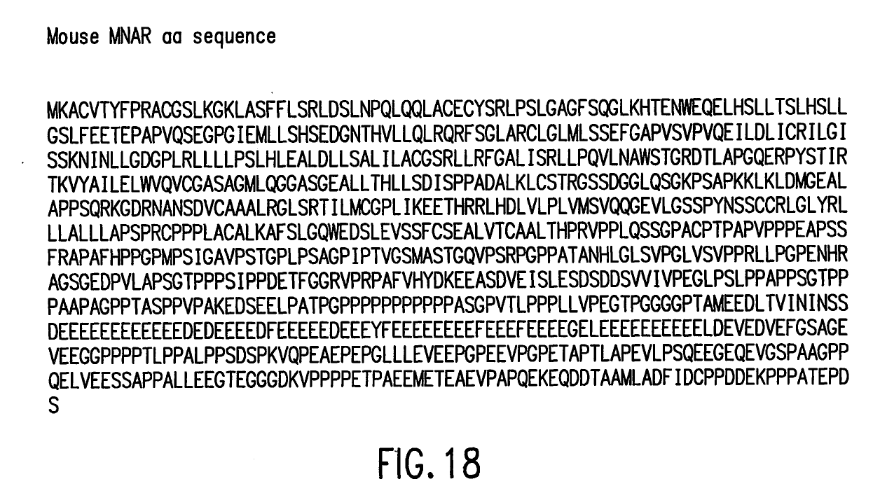

While MNAR utilizes the LXXLL motifs to interact with estrogen receptors, MNAR uses PXXP motifs to interact with Src. MNAR contains three of these PXXP motifs within the N-terminal portion. From the deletion analysis, constructs of MNAR missing the first two PXXP motifs are not able to stimulate estrogen receptor activity in transfection assays (FIG. 13). This suggests these first two PXXP motifs are sufficient for interaction with Src. Mutation of these motifs revealed that the first PXXP motif (aa 55-58) (PXXP mutant 1, SEQ ID NO: 23) is necessary and sufficient to activate ER transcriptional activity, while the second PXXP motif (aa 64-67) (PXXP mutant 2 SEQ ID NO: 32) is dispensable (FIG. 14).

Additional mutants generated in this study were as follows: mutant 5 with aa 1-120 (SEQ ID NO: 24); mutant 6 with aa 1-79 (SEQ ID NO: 25); mutant 7 with aa 1-40 (SEQ ID NO: 26); mutant 8 with aa 190-469 (SEQ ID NO: 27); mutant 12 with aa 888-1131 (SEQ ID NO: 28); mutant 13 with aa 80-594 (SEQ ID NO: 29); and mutant 14 with aa 80-574 (SEQ ID NO: 30). Each of these mutants, along with the other mutants listed above, were schematically diagrammed in FIG. 12 and were analyzed for their ability to enhance ER-stimulated transcription when transfected into HepG2 cells. (FIG. 13)

In conclusion, we demonstrate that interaction of estrogen receptors α and β with MNAR is enhanced by E2 but not by 4(OH) tamoxifen. We also show that the MNAR-ER complex interacts with members of Src family of tyrosine kinases—p60^{src} (Src) and p56^{lck} (Lck) dramatically stimulating Src enzymatic activity. We also show that MNAR, through activation of the Src phosphorylation cascade, affects ER transcriptional activity and ultimately ER-mediated gene expression. Specifically, we demonstrate that short term treatment of MCF-7 cells with E2 leads to MNAR-Src-ER complex formation, activation of Src and phosphorylation of some endogenous proteins. Enhanced MNAR expression in transiently transfected cells leads to stimulation of ER transcriptional activity. Since specific inhibitors of Src and MEK kinases block the ability of MNAR to augment ER transcriptional activity, we conclude that this activation of ER is due to stimulation of the MAP kinase phosphorylation cascade promoted by activation of Src kinases. Alternatively, depletion of MNAR from MCF-7 cells using antisense oligonucleotides inhibits ER-mediated gene expression, supporting its involvement in ER-regulated gene expression. These data reveal that MNAR modulates ER crosstalk with Src family tyrosine kinases and by doing so mediates the non-genomic activity of estrogen receptors.

These studies demonstrate the importance of MNAR in interacting with nuclear receptors. MNAR provides a means of developing compounds that selectively modulate genomic versus non-genomic activity of nuclear receptors.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### REFERENCES

Anzick, S. L., Kononen, J., Walker, R. L., Azorsa, D. O., Tanner, M. M., Guan, X. Y., Sauter, G., Kallioniemi, O. P., Trent, J. M., and Meltzer, P. S. (1997). AlB1, a steroid receptor coactivator amplified in breast and ovarian cancer. Science 277, 965-8.
Aronica S M, W. L. Kraus, B. S. Katzenellenbogen, Proceedings of the National Academy of Sciences of the United States of America 91, 8517-21 (1994).
Bression, D., Michard, M., Le Dafniet, M., Pagesy, P., and Peillon, F. (1986). Evidence for a specific estradiol binding site on rat pituitary membranes. Endocrinology 119, 1048-51.
Bunone G., P. Briand, R. Miksicek, D. Picard, EMBO J. 15, 2174-2183 (1996).
Cantley L C, et al., Cell 64, 281-302 (1991).
Castoria G., et al., EMBO J. 18, 2500-2510 (1999).
Chen, Z., Yuhanna, I. S., Galcheva-Gargova, Z., Karas, R. H., Mendelsohn, M. E., and Shaul, P. W. (1999). Estrogen receptor alpha mediates the nongenomic activation of endothelial nitric oxide synthase by estrogen. J Clin Invest 103, 401-6.
Cheng, H., Nishio, H., Hatase, O., Ralph, S., and Wang, J. (1992). A synthetic peptide derived from p34cdc2 is a specific and efficient substrate of src-family tyrosine kinases. J. Biol. Chem. 267, 9248-9256.
Collins, P., and Webb, C. (1999). Estrogen hits the surface. Nature Medicine 5, 1130-1131.
Cooper, J. A., and Howell, B. (1993). The when and how of Src regulation. Cell 73, 1051-4.
Devergne, O., Hummel, M., Koeppen, H., Le Beau, M., Nathanson, E., Kieff, E., and Birkenbach, M. (1996). A novel interleukin-12 p40-related protein induced by latent Epstein-Barr virus infection in B lymphocytes [published erratum appears in J Virol 1996 April; 70(4):2678]. J. Virol. 70, 1143-1153.
Downward J, Current Biology 7, R258-60 (1997).
Endoh, H., Sasaki, H., Maruyama, K., Takeyama, K., Waga, I., Shimizu, T., Kato, S., and Kawashima, H. (1997). Rapid activation of MAP kinase by estrogen in the bone cell line. Biochem Biophys Res Commun 235, 99-102.
Evans R M 1988 The steroid and thyroid hormone receptor superfamily. Science 240:889-895
Feng W, et al., Mol. Endocrinol 15, 32-45 (2001).
Filardo, EJ, J. A. Quinn, K. I. Bland, A. R. Frackelton, Jr., Mol. Endocrinol 14, 1649-1660 (2000).
Franz, Mueller, Haartong, Frey and Katus (1997). Transgenic animal models: new avenues in cardiovascular disease physiology. J. Mol. Med. 75(2):115-119.
Glass, CK 1994 Differential recognition of target genes by nuclear receptor monomers, dimers, and heterodimers. Endocrine Reviews 15:391-407.
Goetz, R. M., Thatte, H. S., Prabhakar, P., Cho, M. R., Michel, T., and Golan, D. E. (1999). Estradiol induces the calcium-dependent translocation of endothelial nitric oxide synthase. PNAS 96, 2788-2793.
Heery, D. M., Kalkhoven, E., Hoare, S., and Parker, M. G. (1997). A signature motif in transcriptional co-activators mediates binding to nuclear receptors [see comments]. Nature 387, 733-6.
Hong, H., Kohli, K., Garabedian, M. J., and Stallcup, M. R. (1997). GRIP1, a transcriptional coactivator for the AF-2 transactivation domain of steroid, thyroid, retinoid, and vitamin D receptors. Molecular & Cellular Biology 17, 2735-44.
Houthaeve T, G. H., Ashman K, Nillson T, Mann M. (1997). J Protein Chem 16, 343-348.
Hubbard S R, M. Mohammadi, J. Schlessinger, Journal of Biological Chemistry 273, 11987-90 (1998).
Ishii, T., Yanagawa, T., Kawane, T., Yuki, K., Seita, J., Yoshida, H., and Bannai, S. (1996). Murine peritoneal macrophages induce a novel 60-kDa protein with structural similarity to a tyrosine kinase p56lck-associated protein in response to oxidative stress. Biochem Biophys Res Commun 226, 456-60.
Joung, I., Strominger, J. L., and Shin, J. (1996). Molecular cloning of a phosphotyrosine-independent ligand of the p56lck SH2 domain. PNAS 93, 5991-5995.
Kato S, et al., Science 270, 1491-4 (1995).
Katzenellenbogen J A, O'Malley B W, Katzenellenbogen B S 1996 Tripartite steroid hormone receptor pharmacology: interaction with multiple effector sites as a basis for the cell- and promoter-specific action of these hormone. Mol Endocrinol 10:119-131.
Kristen D. Brubaker, C. V. G. (1999). Estrogen stimulates protein tyrosine phosphorylation and Src kinase activity in avian osteoclasts. Journal of Cellular Biochemistry Volume 76, Issue 2, 2000. P 76, 206-216.
Mangelsdorf, D. J., Thummel, C., Beato, M., Herrlich, P., Schutz, G., Umesono, K., Blumberg, B., Kastner, P., Mark, M., Chambon, P., and et al. (1995). The nuclear receptor superfamily: the second decade. Cell 83, 835-9.
Marcus, S. L., Winrow, C. J., Capone, J. P., and Rachubinski, R. A. (1996). A p561ck Ligand Serves as a Coactivator of an Orphan Nuclear Hormone Receptor. J. Biol. Chem. 271, 27197-27200.
Marshall C J, Curr Opin Cell Biol 8, 197-204 (1996).
Matsuda M, B. J. Mayer, Y. Fukui, H. Hanafusa, Science 248, 1537-9 (1990).
McKenna, N.J., Lanz, R. B., and O'Malley, B. W. (1999). Nuclear receptor coregulators: cellular and molecular biology. Endocrine Reviews 20, 321-44.
Migliaccio, A., Castoria, G., Di Domenico, M., de Falco, A., Bilancio, A., Lombardi, M., Barone, M. V., Ametrano, D., Zannini, M. S., Abbondanza, C., and Auricchio, F. (2000). Steroid-induced androgen receptor-oestradiol receptor {beta}-Src complex triggers prostate cancer cell proliferation. EMBO J. 19, 5406-5417.
Migliaccio, A., Di Domenico, M., Castoria, G., de Falco, A., Bontempo, P., Nola, E., and Auricchio, F. (1996). Tyrosine kinase/p21 ras/MAP-kinase pathway activation by estradiol-receptor complex in MCF-7 cells. EMBO J. 15, 1292-1300.
Migliaccio, A., Pagano, M., and Auricchio, F. (1993). Immediate and transient stimulation of protein tyrosine phosphorylation by estradiol in MCF-7 cells. Oncogene 8, 2183-91.
Migliaccio, A., Piccolo, D., Castoria, G., Di Domenico, M., Bilancio, A., Lombardi, M., Gong, W., Beato, M., and Auricchio, F. (1998). Activation of the Src/p21ras/ERk pathway by progesterone receptor via cross-talk with estrogen receptor. EMBO J. 17, 2008-2018.
Oursler, M. J. (1998). Estrogen regulation of gene expression in osteoblasts and osteoclasts. Critical Reviews in Eukaryotic Gene Expression 8, 125-40.
Pappas, T. C., Gametchu, B., and Watson, C. S. (1995). Membrane estrogen receptors identified by multiple antibody labeling and impeded-ligand binding. FASEB Journal 9, 404-10.
Park, I., Chung, J., Walsh, C., Yun, Y., Strominger, J., and Shin, J. (1995). Phosphotyrosine-Independent Binding of a 62-kDa Protein to the src Homology 2 (SH2) Domain of p56lck and Its Regulation by Phosphorylation of Ser-59 in the Ick Unique N-Terminal Region. PNAS 92, 12338-12342.
Parker, M. G., and White, R. (1996). Nuclear receptors spring into action [news; comment]. Nature Structural Biology 3, 113-5.
Pietras, R. J., and Szego, C. M. (1979). Metabolic and proliferative responses to estrogen by hepatocytes selected for plasma membrane binding-sites specific for estradiol-17beta. Journal of Cellular Physiology 98, 145-59.
Pietras, R. S., C (1975). Endometrial cell calcium and oestrogen action. Nature 253, 357-359.
Pietras, R. S.C. (1977). Specific binding sites for estrogen at the outer surfaces of isolated endometrial cells. Nature 265, 69-72.
Puls, A., Schmidt, S., Grawe, F., and Stabel, S. (1997). Interaction of protein kinase C zeta with ZIP, a novel protein kinase C-binding protein. PNAS 94, 6191-6196.
Razandi M, A. Pedram, G. L. Greene, E. R. Levin, Molecular Endocrinology 13, 307-19 (1999).
Revelli A, M. Massobrio, J. Tesarik, Endocr Rev 19, 3-17 (1998).
Sambrook, Fritsch and Maniatis. Molecular Cloning—A Laboratory Manual. Cold Spring Press (Cold Spring Harbor, N.Y.). 1989.
Simoncini T., H.-M. A., Brazil D. P., Ley K., Chin W. W.&, and J. K., L. (2000). Interaction of oestrogen receptor with the regulatory subunit of phosphatidylinositol-3-OH kinase. Nature 407, 538-541.
Singer, C. A., Figueroa-Masot, X. A., Batchelor, R. H., and Dorsa, D. M. (1999). The mitogen-activated protein kinase pathway mediates estrogen neuroprotection after glutamate toxicity in primary cortical neurons. J Neurosci 19, 2455-63.
Torchia, J., Rose, D. W., Inostroza, J., Kamei, Y., Westin, S., Glass, C. K., and Rosenfeld, M. G. (1997). The transcriptional co-activator p/CIP binds CBP and mediates nuclear-receptor function [see comments]. Nature 387, 677-84.
Thomas, S M, Brugge, J S Annual Review of Cell & Developmental Biology 13, 513-609 (1997).
Tsai M-J, O'Malley B W 1994 Molecular mechanisms of action of steroid/thyroid receptor superfamily members. Annu Rev Biochem 63:451-486.
Voegel, J. J., Heine, M. J., Tini, M., Vivat, V., Chambon, P., and Gronemeyer, H. (1998). The coactivator TIF2 contains three nuclear receptor-binding motifs and mediates transactivation through CBP binding-dependent and -independent pathways. EMBO Journal 17, 507-19.
Weiss, A., and Littman, D. R. (1994). Signal transduction by lymphocyte antigen receptors. Cell 76, 263-74.

1. An isolated nucleic acid sequence comprising
a polynucleotide selected from the group consisting of:
(a) a polynucleotide encoding a novel protein modulator of non-genomic activity of nuclear receptors (MNAR) comprising the amino acid sequence of SEQ ID NO:2 or of SEQ ID NO:13;
(b) a polynucleotide that hybridizes under highly stringent conditions with (i) a region of the nucleotide sequence of SEQ ID NO: 1 or of SEQ ID NO:12, (ii) a subsequence of at least 100 nucleotides of the nucleotide sequence of SEQ ID NO: 1 or of SEQ ID NO:12, (iii) or a complementary strand of (i) or (ii);
(c) a polynucleotide comprising a sequence with at least 85% identity to a polynucleotide coding sequence of SEQ ID NO: 1 or of SEQ ID NO: 12;
(d) a variant of the polynucleotide comprising a polynucleotide coding sequence of SEQ ID NO:1 or of SEQ ID NO:12; and
(e) a polynucleotide encoding a polypeptide fragment comprising the amino acids of SEQ ID NOS: 20 or 23
(f) a complement or antisense form of the polynucleotide of (a), (b), (c), (d) or (e);
wherein the polynucleotide does not encode a p160 protein:

2. The isolated nucleic acid sequence of claim 1, wherein
the polynucleotide sequence is an allelic variant.

3. A polypeptide comprising an amino acid sequence selected from the group consisting of:
(a) an amino acid sequence for a protein modulator of non-genomic activity of nuclear receptors (MNAR) comprising the amino acid sequence of SEQ ID NO: 2 or of SEQ ID NO:13;
(b) an amino acid sequence for an MNAR fragment comprising the amino acid sequence of SEQ ID NOS:14, 15 or 16;
(c) a variant of (a) or (b), and
(d) a fragment of (a), (b), or (c).

4. The polypeptide of claim 3 comprising one or more amino acid sequences which are more than 90% identical to the amino acid sequence of SEQ ID NO:14.

5. The polypeptide of claim 3 comprising one or more amino acid sequences which are more than 95% identical to the mutant sequence of SEQ ID NO:14.

6. The polypeptide of claim 3, wherein said polypeptide comprises the amino acid sequence of SEQ ID NOS 2, 13, 14, 15 16 20 or 22.

7. A fusion polypeptide comprising a polypeptide, or polypeptide fragment of MNAR.

8. The polypeptide according to claim 3, wherein said polypeptide stimulates the activity of a tyrosine kinase in the presence of one or more nuclear receptors.

9. The polypeptide of claim 8, wherein said nuclear receptor is a steroid receptor.

10. The polypeptide of claim 8, wherein said nuclear receptor is an estrogen receptor.

11. The polypeptide of claim 8, wherein said estrogen receptor is estrogen receptor alpha.

12. An expression vector comprising the isolated nucleic acid sequence of claim 1.

13. The expression vector of claim 12 comprising the polynucleotide sequence of SED ID NO: 1 or of SEQ ID NO:12.

14. A host cell transformed with the vector of claim 12.

15. The host cell of claim 14, wherein the host cell is a mammalian host cell.

16. An isolated antibody to the polypeptide of claim 3.

17. The antibody of claim 16, wherein said antibody is a monoclonal antibody.

18. The antibody of claim 16, wherein said antibody is a polyclonal antibody.

19. A method for modulating the transcriptional activity of
a nuclear receptor, comprising providing to the loci of a nuclear receptor a polypeptide according to claim 3.

20. A method for modulating the transcriptional activity of
a nuclear receptor, comprising:
(a) transforming a host cell with the recombinant vector having the nucleic acid sequence for MNAR according to claim 1; and
(b) culturing said transformed host cell to express the nucleic acid in the presence of a nuclear receptor.

21. A method of identifying compounds exhibiting non-genomic activity versus genomic activity comprising administering a test compound to a cell comprising an MNAR-nuclear receptor complex and measuring the non-genomic or genomic activity resulting from the test compound.

22. The method of claim 21, wherein at least one ligand of ER is present in the cell, or a kinase is present, or both are present.

23. A method of screening for a compound that modulates the activity of MNAR on a nuclear receptor, comprising the steps of (a) contacting a test compound with the polypeptide of claim 3; and (b) determining whether said test compound specifically binds said polypeptide.

24. A method of screening for a compound that modulates the activity of MNAR on a nuclear receptor, said method comprising the steps of (a) adding a test compound to a cell comprising the polypeptide of claim 3 and a nuclear receptor; and (b) comparing the MNAR activity before and after said adding step.

25. The method of claim 24, said method comprising adding a test compound to a control comprising a mutant cell lacking MNAR activity or with significantly reduced MNAR activity.

26. A method for detecting the non-genomic activity of a compound comprising administering a compound to a cell in the presence of MNAR-nuclear receptor and in the absence of MNAR and comparing the level of non-genomic activity.

27. A method for detecting the genomic activity of a compound comprising administering a compound to a cell comprising a MNAR-nuclear receptor complex and comparing the level of genomic activity in the absence of MNAR.

28. A method for detecting the genomic activity of a compound according to claim 51 wherein the MNAR-ER cell overexpresses MNAR.

29. A method for detecting the genomic activity of a compound according to claim 51 wherein the MNAR-ER cell overexpresses ER.

30. A method for detecting the genomic activity of a compound according to claim 27 wherein the ER is operatively associated with a reporter.

31. A method of identifying compounds having selective genomic versus non-genomic activity said method comprising the steps of (a) adding a test compound to a cell comprising a MNAR-nuclear receptor complex; and (b) comparing the genomic versus non-genomic activity before and after said adding the test compound.

32. The method of claim 59, wherein selective genomic activity is measured as a two-fold increase after addition of said test compound to a cell in the presence of MNAR-nuclear receptor when compared to genomic activity with test compound in the absence of MNAR and wherein no change is observed in non-genomic activity after addition of said test compound.

33. The method of claim 31, wherein non-genomic activity is increased two-fold after addition of said test compound to a cell in the presence of MNAR-nuclear receptor complex when compared to non-genomic activity with test compound in the absence of MNAR and wherein no change is observed in genomic activity after addition of said test compound.

34. A method of identifying compounds that modulate the non-genomic activity of a nuclear receptor, comprising determining an increase or decrease in the transcriptional activity of a nuclear receptor in the presence of MNAR.

35. A method of determining whether a desired phenotype of cell is effected by non-genomic activity or genomic activity of a nuclear receptor comprising determining the effect of a selected phenotype of cell is increased or decreased when the cell is exposed to additional amounts of MNAR; and then detecting the presence or absence of a desired phenotype, wherein the absence of a desired activity or phenotype, in the presence of additional MNAR indicates increased non-genomic activity of a nuclear receptor negatively affects the desired phenoype (i.e. the non-genomic activity is not beneficial to said cell) and wherein the increase of a desired phenotype indicates that increased non-genomic activity of a nuclear receptor positively affects said cell.

36. A method according to claims 2, wherein the cell employed is cell that is positively affected by non-genomic activity of the nuclear receptor.

37. A method according to claims 21, wherein the cell employed is cell that is negatively affected by non-genomic activity of the nuclear receptor.

38. A method of identifying compounds that modulate the non-genomic activity of a nuclear receptor, comprising determining an increase or decrease in the transcriptional activity of said nuclear receptor and determining whether a kinase, affected by the presence of MNAR and said nuclear receptor, has an increase or decrease in enzymatic activity.

39. The method of claim 19, wherein the nuclear receptor is a steroid receptor.

40. The method of claim 19, wherein the nuclear receptor is a non-steroid receptor.

41. The method of claim 19, wherein the nuclear receptor is an orphan receptor.

42. The method of claim 19, wherein the steroid receptor is an estrogen receptor.

43. A non-human transgenic mammal, comprising one or more cells
which comprise at least one non-functional endogenous MNAR polynucleotide sequence.

44. The non-human transgenic mammal of claim 43, wherein said mammal is selected from the group consisting of a mouse, a rat, a hamster, a guinea pig, a rabbit, a cow, and a sheep.

45. The non-human transgenic mammal of claim 43, comprising at least one non-functional endogenous nuclear receptor.

46. The non-human transgenic mammal of claim 43, wherein the non-functional endogenous receptor is a steroid receptor.

47. The non-human transgenic mammal of claim 43, wherein the non-functional endogenous receptor is a non-steroid receptor.

48. The non-human transgenic mammal of claim 43, wherein the non-functional endogenous receptor is an orphan receptor.

49. The non-human transgenic mammal of claim 43, wherein said the non-functional endogenous receptor is an estrogen receptor.

50. The non-human transgenic mammal of claim 49, wherein endogenous forms of ERα and ERβ are non-functional.

51. The non-human transgenic mammal of claim 43, comprising an interruption of the MNAR coding sequence.

52. The non-human transgenic mammal of claim 49, comprising an interruption of the MNAR and ER coding sequences.

53. The non-human transgenic mammal of claim 43, comprising a nonsense MNAR mutation.

54. The non-human transgenic mammal of claim 49, comprising a nonsense mutations of MNAR and ER.

55. The non-human transgenic mammal of claim 43, comprising a deletion of MNAR coding sequences.

56. The non-human transgenic mammal of claim 43, comprising an alteration in the regulatory region of the MNAR gene making MNAR non-functional.

57. The non-human transgenic mammal of claim 56, comprising substitution of an inducible/repressable promoter for the endogenous MNAR promoter.

58. The non-human transgenic mammal of claim 57, comprising at least one inducible/repressable promoter replacing the endogenous MNAR promoter and the estrogen receptor.

59. The non-human transgenic mammal of claim 43, wherein cells of said mammal further comprise an exogenous selectable marker gene under the control of a promoter active in at least one cell type of said mammal.

60. A method of screening for a compound that disrupts the function
of MNAR, comprising using a protein-protein interaction assay to identify compounds that interact with MNAR.

61. A method of screening for a compound that disrupts the function
of MNAR, comprising using a two-hybrid assay to identify proteins that interact with MNAR.

62. The method of claim 61 wherein the two-hybrid is conducted in yeast or mammalian cells.

63. A process for producing a polypeptide of claim 3 comprising:
(a) culturing a host cell transformed with a nucleic acid sequence for MNAR according to claim 1 under suitable culture medium under conditions for growth; and
(b) isolating the polypeptide from the culture medium.
(c)

64. A polypeptide produced from the process of claim 64.


SEQUENCE LISTING (33 sequences)

SEQ ID NO: 1 - DNA, 3391 bp, Homo sapiens
atggcggcagccgttctgagtgggccctctgcgggctccgcggctggggttcctggcgggaccgggggtctctcggcagtgagctcgggcccgcggctccgcctgctgctgctggagagtgtttctggtttgctgcaacctcgaacggggtctgccgttgctccggtgcatcccccaaaccgctcggccccacatttgcccgggctcatgtgcctattgcggctgcatgggtcggtgggcggggcccagaacctttcagctcttggggcattggtgagtctcagtaatgcacgtctcagttccatcaaaactcggtttgagggcctgtgtctgctgtccctgctggtaggggagagccccacagagctattccagcagcactgtgtgtcttggcttcggagcattcagcaggtgttacagacccaggacccgcctgccacaatggagctggccgtggctgtcctgagggacctcctccgatatgcagcccagctgcctgcactgtccgggacatctccatgaaccacctccctggccttctcacctccctgctgggcctcaggccagagtgtgagcagtcagcattggaaggaatgaaggcttgtatgacctatttccctcgggcttgtggttctctcaaaggcaagctggcctcattttttctgtctagggtggatgccttgagccctcagctccaacagttggcctgtgagtgttattcccggctgccctctttaggggctggcttttcccaaggcctgaagcacaccgagagctgggagcaggagctacacagtctgctggcctcactgcacaccctgctgggggccctgtacgagggagcagagactgctcctgtgcagaatgaaggccctggggtggagatgctgctgtcctcagaagatggtgatgcccatgtccttctccagcttcggcagaggttttcgggactggcccgctgcctagggctcatgctcagctctgagtttggagctcccgtgtccgtccctgtgcaggaaatcctggatttcatctgccggaccctcagcgtcagtagcaagaatattagcttgcatggagatggtcccctgcggctgctgctgctgccctctatccaccttgaggccttggacctgctgtctgcactcatcctcgcgtgtggaagccggctcttgcgctttgggatcctgatcggccgcctgcttccccaggtcctcaattcctggagcatcggtagagattccctctctccaggccaggagaggccttacagcacggttcggaccaaggtgtatgcgatattagagctgtgggtgcaggtttgtggggcctcggcgggaatgcttcagggaggagcctctggagaggccctgctcacccacctgctcagcgacatctccccgccagctgatgcccttaagctgcgtagcccgcgggggagccctgatgggagtttgcagactgggaagcctagcgcccccaagaagctaaagctggatgtgggggaagctatggccccgccaagccaccggaaaggggatagcaatgccaacagcgacgtgtgtgcggctgcactcagaggcctcagccggaccatcctcatgtgtgggcctctcatcaaggaggagactcacaggagactgcatgacctggtcctccccctggtcatgggtgtacagcagggtgaggtcctaggcagctccccgtacacgagctcccgctgccgccgtgaactctactgcctgctgctggcgctgctgctggccccgtctcctcgctgcccacctcctcttgcctgtgccctgcaagccttctccctcggccagcgagaagatagccttgaggtctcctctttctgctcagaagcactggtgacctgtgctgctctgacccacccccgggttcctcccctgcagcccatgggccccacctgccccacacctgctccagttccccctcctgaggccccatcgcccttcagggccccaccgttccatcctccgggccccatgccctcagtgggctccatgccctcagcaggccccatgccctcagcaggccccatgccctcagcaggccctgtgccctcggcacgccctggacctcccaccacagccaaccacctaggcctttctgtcccaggcctagtgtctgtccctccccggcttcttcctggccctgagaaccaccgggcaggctcaaatgaggaccccatccttgcccctagtgggactcccccacctactatacccccagatgaaacttttggggggagagtgcccagaccagcctttgtccactatgacaaggaggaggcatctgatgtggagatctccttggaaagtgactctgatgacagcgtggtgatcgtgcccgaggggcttccccccctgccacccccaccaccctcaggtgccacaccaccccctatagcccccactgggccaccaacagcctcccctcctgtgccagcgaaggaggagactgaagaacttcctgcagccccagggcctctcccgccacccccacctccgccgccgcctgttcctggtcctgtgacgctccctccaccccagttggtccctgaagggactcctggtgggggaggacccccagccctggaagaggatttgacagttattaatatcaacagcagtgatgaagaggaggaggaagaggaagaaggggaagaagaagaagaggaagaagaggaagaggaggaagactttgaggaagaggaagaggatgaagaggaatattttgaagaggaagaagaggaggaagaagagtttgaggaagaatttgaggaagaagaaggtgagttagaggaagaagaagaagaggaggatgaggaggaggaagaagaactggaagaggtggaagacctggagtttggcacagcaggaggggaggtagaagaaggtgcacctccacccccaaccctgcctccagctctgcctccccctgagtctcccccaaaggtgcagccagaacccgaacccgaacccgggctgcttttggaagtggaggagccagggacggaggaggagcgtggggctgacacagctcccaccctggcccctgaagcgctcccctcccagggagaggtggagagggaaggggaaagccctgcggcagggccccctccccaggagcttgttgaagaagagccctctgctcccccaaccctgttggaagaggagcctgaggatgggagtgacaaggtgcagcccccaccagagacacctgcagaagaagaatggagacagagacagaggccgaagctctccaggaaaaggagcaggatgacacagctgccatgctggccgacttcatcgattgtccccctgatgatgagaagccaccacctcccacagagcctgactcctag

SEQ ID NO: 2 - protein, 1130 aa, Homo sapiens
MAAAVLSGPSAGSAAGVPGGTGGLSAVSSGPRLRLLLLESVSGLLQPRTGSAVAPVHPPNRSAPHLPGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPPADALKLRSPRGSPDGSLQTGKPSAPKKLKLDVGEAMAPPSHRKGDSNANSDVCAAALRGLSRTILMCGPLIKEETHRRLHDLVLPLVMGVQQGEVLGSSPYTSSRCRRELYCLLLALLLAPSPRCPPPLACALQAFSLGQREDSLEVSSFCSEALVTCAALTHPRVPPLQPMGPTCPTPAPVPPPEAPSPFRAPPFHPPGPMPSVGSMPSAGPMPSAGPMPSAGPVPSARPGPPTTANHLGLSVPGLVSVPPRLLPGPENHRAGSNEDPILAPSGTPPPTIPPDETFGGRVPRPAFVHYDKEEASDVEISLESDSDDSVVIVPEGLPPLPPPPPSGATPPPIAPTGPPTASPPVPAKEEPEELPAAPGPLPPPPPPPPPVPGPVTLPPPQLVPEGTPGGGGPPALEEDLTVININSSDEEEEEEEEGEEEEEEEEEEEEDFEEEEEDEEEYFEEEEEEEEEFEEEFEEEEGELEEEEEEEDEEEEEELEEVEDLEFGTAGGEVEEGAPPPPTLPPALPPPESPPKVQPEPEPEPGLLLEVEEPGTEEERGADTAPTLAPEALPSQGEVEREGESPAAGPPPQELVEEEPSAPPTLLEEEPEDGSDKVQPPPETPAEEEMETETEAEALQEKEQDDTAAMLADFIDCPPDDEKPPPPSEPDS

SEQ ID NO: 3 - DNA, 30 bp, Homo sapiens
taggatccagatggcggcagccgttctgag

SEQ ID NO: 4 - DNA, 18 bp, Homo sapiens
cgatcaggatcccaaagc

SEQ ID NO: 5 - DNA, 18 bp, Homo sapiens
gctttgggatcctgatcg

SEQ ID NO: 6 - DNA, 18 bp, Homo sapiens
caaggagatctccacatc

SEQ ID NO: 7 - DNA, 18 bp, Homo sapiens
gatgtggagatctccttg

SEQ ID NO: 8 - DNA, 18 bp, Homo sapiens
gctaggagtcaggctctg

SEQ ID NO: 9 - DNA, 33 bp, Homo sapiens
ccgaagccaagacacacagtgctgctggaatag

SEQ ID NO: 10 - DNA, 30 bp, Homo sapiens
ctggagaaaaaaggggcagagataaagagt

SEQ ID NO: 11 - DNA, 25 bp, Homo sapiens
catggagatgtcccggaacagtgca

SEQ ID NO: 12 - DNA, 3435 bp, Murinae gen. sp.
ggctcgagcctgtgaagatggcggcggccgttcttagtggggcctctgcgggctccccagctggggctcctggcgggcccgggggtctctctgcggtgggatccggcccgcgtctgcgtctgctgctgttagagagcatttccgggttgttacagccgcgaacggcgtcccccgtggcccctgtgcatcccccaatccagtgggccccgcatttgcccgggctcatgtgcttattgaggctgcatgggacggcaggtggggcgcaaaatctttcagctcttggggcattggtgaatctcagtaatgcacatcttggctccatcaaaactcggtttgaaggcctgtgtctgctgtccttgttgataggggagagccccacagagttattccagcaacattgtgtttcttggcttcggagcatccagcaggtgctgcagtctcaggactcaccgtccaccatggagctggctgtggctgtcctgagggatctgctccgacatgcatcccagcttcctaccctgtttcgggacatctccaccaaccaccttcctgggcttcttacttccttgctgggcctcagaccagagtgtgaacaatcagctttggagggaatgaaggcttgtgtgacctattttcctcgggcctgtggctctctaaaaggaaagctggcttcctttttcctgtctcgattggattccctgaaccctcagctccaacagctggcctgcgagtgttactccaggttgccctcgttaggtgctggcttttcccagggactgaagcacacagagaactgggaacaggagctgcacagcctgctgacctcactgcacagcttactggggtccctgtttgaggaaacagagcctgctcctgtgcagagtgaaggccctggaatagagatgctgctttcccactcagaagatggtaacacccatgtcctcctccagcttcggcagaggttttcaggcctggcccgttgcctggggctcatgctcagctctgagtttggggctcctgtgtccgttcctgtgcaggaaatcctggacctcatctgccggatccttggtatcagtagcaagaatattaacttgcttggagatggtcccctcaggttgctgctgctgccttctctgcaccttgaggccttggacttgctctctgccctgatccttgcgtgtggaagtcgacttttgcgttttggtgccctgatcagccggctgcttccccaggttctcaatgcctggagcactgggagggatactctagctccaggccaggagaggccttacagcaccattcggaccaaagtgtatgctatcttagagctatgggtgcaggtttgtggggcctcagcaggtatgcttcagggaggagcctctggagaagccttgctcacccacctgctcagtgacatctcccctccagctgatgctcttaagctatgcagtaccaggggaagctctgatggaggtttgcaaagtgggaagccaagtgctcctaagaagctcaagctcgatatgggggaagctttggctccacccagccaaaggaaaggagataggaatgccaacagtgacgtgtgcgcagctgcactgagaggcctcagtcggaccatccttatgtgcgggcctctcatcaaggaggagactcacaggagacttcatgatctagtcctgcccctggtcatgagtgtccagcagggtgaggtcctgggcagctctccttacaacagttcctgctgtcgcctcgggctctaccgcctgctgctggctttattgctggcaccttcccctcggtgccctcctcctcttgcctgtgccctgaaagctttctctcttggccaatgggaagatagtcttgaggtctcctctttttgctcagaagctctggtgacctgtgctgctctgacacacccccgagtacctcctctacagagctcaggccctgcctgccccactcctgcccccgtccctcctcctgaggccccatcttcattcagggccccggccttccatcctccaggccccatgccctccataggtgctgtgccatccacaggcccattaccctcagcaggccccattcccacagtaggctctatggcgtccacaggccaagtgccctctcgccctggacctccagcaacagccaaccatttaggcctctctgtcccaggcctggtatctgttccacccaggctccttcctggccctgaaaaccaccgtgcaggctccggtgaggatcctgtccttgctcctagtgggacccctccgcctagcatacctccagatgaaacttttggggggagagtacctaggccagcctttgtccactacgataaggaggaggcatctgatgtagagatctccttggaaagcgactctgatgacagtgtggtgattgtacctgaggggcttccctccctgccacctgcaccaccctcaggcactcctccccctgcggctcctgccggcccaccaacagcctcacctcctgtgccagctaaggaggattctgaagaactgcctgcaaccccagggcctccacccccacccccacccccacccccaccggcctctggtcctgtgacacttcctccacccctgctggtccctgaagggactcctggagggggaggacccacagccatggaagaagatttgacagttattaatatcaacagcagtgatgaagaggaggaggaggaagaggaagaggaggaagaagatgaagatgaggaagaagaagactttgaggaagaggaagaagatgaagaagaatattttgaagaggaagaagaggaagaagagtttgaggaggaatttgaggaggaagaaggtgaattagaagaggaagaagaagaggaggaagaagagttagatgaggtagaagatgtggagtttggttcagcaggggaggtggaagaaggtggacctccacccccaacactgccacctgctctgccaccttcagactcgcccaaagtacagcctgaggcagaacctgaacctgggctcttattggaagttgaggagccagggccagaggaggtgcctgggcctgagactgctcctaccctggcccctgaagtgctcccctcccaggaggagggggagcaggaagtgggaagccctgcagcagggccacctcaggagcttgttgaagagtcctctgctccccctgccctgctggaagaggggactgagggtggaggtgacaaagtgccacccccaccggagacacctgcagaagaaatggagacagaagcagaagttccagctcctcaggaaaaggagcaagatgatacagctgccatgctggctgatttcatcgattgtccccctgacgatgagaagccaccacctgctacagagcctgactcgtagccccttctgcaccccgtcactttgtatccaataaagttctgtctgt

SEQ ID NO: 13 - protein, 925 aa, Murinae gen. sp.
MKACVTYFPRACGSLKGKLASFFLSRLDSLNPQLQQLACECYSRLPSLGAGFSQGLKHTENWEQELHSLLTSLHSLLGSLFEETEPAPVQSEGPGIEMLLSHSEDGNTHVLLQLRQRFSGLARCLGLMLSSEFGAPVSVPVQEILDLICRILGISSKNINLLGDGPLRLLLLPSLHLEALDLLSALILACGSRLLRFGALISRLLPQVLNAWSTGRDTLAPGQERPYSTIRTKVYAILELWVQVCGASAGMLQGGASGEALLTHLLSDISPPADALKLCSTRGSSDGGLQSGKPSAPKKLKLDMGEALAPPSQRKGDRNANSDVCAAALRGLSRTILMCGPLIKEETHRRLHDLVLPLVMSVQQGEVLGSSPYNSSCCRLGLYRLLLALLLAPSPRCPPPLACALKAFSLGQWEDSLEVSSFCSEALVTCAALTHPRVPPLQSSGPACPTPAPVPPPEAPSSFRAPAFHPPGPMPSIGAVPSTGPLPSAGPIPTVGSMASTGQVPSRPGPPATANHLGLSVPGLVSVPPRLLPGPENHRAGSGEDPVLAPSGTPPPSIPPDETFGGRVPRPAFVHYDKEEASDVEISLESDSDDSVVIVPEGLPSLPPAPPSGTPPPAAPAGPPTASPPVPAKEDSEELPATPGPPPPPPPPPPPASGPVTLPPPLLVPEGTPGGGGPTAMEEDLTVININSSDEEEEEEEEEEEEDEDEEEEDFEEEEEDEEEYFEEEEEEEEFEEEFEEEEGELEEEEEEEEEELDEVEDVEFGSAGEVEEGGPPPPTLPPALPPSDSPKVQPEAEPEPGLLLEVEEPGPEEVPGPETAPTLAPEVLPSQEEGEQEVGSPAAGPPQELVEESSAPPALLEEGTEGGGDKVPPPPETPAEEMETEAEVPAPQEKEQDDTAAMLADFIDCPPDDEKPPPATEPDS

SEQ ID NO: 14 - protein, 469 aa, Homo sapiens
MAAAVLSGPSAGSAAGVPGGTGGLSAVSSGPRLRLLLLESVSGLLQPRTGSAVAPVHPPNRSAPHLPGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPP

SEQ ID NO: 15 - protein, 278 aa, Homo sapiens
MAAAVLSGPSAGSAAGVPGGTGGLSAVSSGPRLRLLLLESVSGLLQPRTGSAVAPVHPPNRSAPHLPGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGAL

SEQ ID NO: 16 - protein, 189 aa, Homo sapiens
MAAAVLSGPSAGSAAGVPGGTGGLSAVSSGPRLRLLLLESVSGLLQPRTGSAVAPVHPPNRSAPHLPGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRP

SEQ ID NO: 17 - protein, 536 aa, Homo sapiens
PPPLACALQAFSLGQREDSLEVSSFCSEALVTCAALTHPRVPPLQPMGPTCPTPAPVPPPEAPSPFRAPPFHPPGPMPSVGSMPSAGPMPSAGPMPSAGPVPSARPGPPTTANHLGLSVPGLVSVPPRLLPGPENHRAGSNEDPILAPSGTPPPTIPPDETFGGRVPRPAFVHYDKEEASDVEISLESDSDDSVVIVPEGLPPLPPPPPSGATPPPIAPTGPPTASPPVPAKEEPEELPAAPGPLPPPPPPPPPVPGPVTLPPPQLVPEGTPGGGGPPALEEDLTVININSSDEEEEEEEEGEEEEEEEEEEEEDFEEEEEDEEEYFEEEEEEEEEFEEEFEEEEGELEEEEEEEDEEEEEELEEVEDLEFGTAGGEVEEGAPPPPTLPPALPPPESPPKVQPEPEPEPGLLLEVEEPGTEEERGADTAPTLAPEALPSQGEVEREGESPAAGPPPQELVEEEPSAPPTLLEEEPEDGSDKVQPPPETPAEEEMETETEAEALQEKEQDDTAAMLADFIDCPPDDEKPPPPSEPDS

SEQ ID NO: 18 - protein, 368 aa, Homo sapiens
PPPLACALQAFSLGQREDSLEVSSFCSEALVTCAALTHPRVPPLQPMGPTCPTPAPVPPPEAPSPFRAPPFHPPGPMPSVGSMPSAGPMPSAGPMPSAGPVPSARPGPPTTANHLGLSVPGLVSVPPRLLPGPENHRAGSNEDPILAPSGTPPPTIPPDETFGGRVPRPAFVHYDKEEASDVEISLESDSDDSVVIVPEGLPPLPPPPPSGATPPPIAPTGPPTASPPVPAKEEPEELPAAPGPLPPPPPPPPPVPGPVTLPPPQLVPEGTPGGGGPPALEEDLTVININSSDEEEEEEEEGEEEEEEEEEEEEDFEEEEEDEEEYFEEEEEEEEEFEEEFEEEEGELEEEEEEEDEEEEEELEEVED

SEQ ID NO: 19 - protein, 293 aa, Homo sapiens
PPPLACALQAFSLGQREDSLEVSSFCSEALVTCAALTHPRVPPLQPMGPTCPTPAPVPPPEAPSPFRAPPFHPPGPMPSVGSMPSAGPMPSAGPMPSAGPVPSARPGPPTTANHLGLSVPGLVSVPPRLLPGPENHRAGSNEDPILAPSGTPPPTIPPDETFGGRVPRPAFVHYDKEEASDVEISLESDSDDSVVIVPEGLPPLPPPPPSGATPPPIAPTGPPTASPPVPAKEEPEELPAAPGPLPPPPPPPPPVPGPVTLPPPQLVPEGTPGGGGPPALEEDLTVININSSD

SEQ ID NO: 20 - protein, 6 aa, Homo sapiens
VLRDLL

SEQ ID NO: 21 - protein, 6 aa, Homo sapiens
HLPGLL

SEQ ID NO: 22 - protein, 5 aa, Homo sapiens
LTSLL

SEQ ID NO: 23 - protein, 469 aa, Homo sapiens
MAAAVLSGPSAGSAAGVPGGTGGLSAVSSGPRLRLLLLESVSGLLQPRTGSAVAAVHAPNRSAPHLPGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPP

SEQ ID NO: 24 - protein, 469 aa, Homo sapiens
MAAAVLSGPSAGSAAGVPGGTGGLSAVSSGPRLRLLLLESVSGLLQPRTGSAVAPVHPPNRSAAHLAGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPP

SEQ ID NO: 25 - protein, 469 aa, Homo sapiens
MAAAVLSGPSAGSAAGVPGGTGGLSAVSSGPRLRLLLLESVSGLLQPRTGSAVAAVHAPNRSAAHLAGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPP

SEQ ID NO: 26 - protein, 120 aa, Homo sapiens
MAAAVLSGPSAGSAAGVPGGTGGLSAVSSGPRLRLLLLESVSGLLQPRTGSAVAPVHPPNRSAPHLPGLMCLLRLHGSVGGAQNLSALGALVSLSNARLSSIKTRFEGLCLLSLLVGESP

SEQ ID NO: 27 - protein, 79 aa, Homo sapiens
MAAAVLSGPSAGSAAGVPGGTGGLSAVSSGPRLRLLLLESVSGLLQPRTGSAVAPVHPPNRSAPHLPGLMCLLRLHGSV

SEQ ID NO: 28 - protein, 40 aa, Homo sapiens
MAAAVLSGPSAGSAAGVPGGTGGLSAVSSGPRLRLLLLES

SEQ ID NO: 29 - protein, 280 aa, Homo sapiens
ECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPP

SEQ ID NO: 30 - protein, 243 aa, Homo sapiens
EEEEEEEEGEEEEEEEEEEEEDFEEEEEDEEEYFEEEEEEEEEFEEEFEEEEGELEEEEEEEDEEEEEELEEVEDLEFGTAGGEVEEGAPPPPTLPPALPPPESPPKVQPEPEPEPGLLLEVEEPGTEEERGADTAPTLAPEALPSQGEVEREGESPAAGPPPQELVEEEPSAPPTLLEEEPEDGSDKVQPPPETPAEEEMETETEAEALQEKEQDDTAAMLADFIDCPPDDEKPPPPSEPDS

SEQ ID NO: 31 - protein, 515 aa, Homo sapiens
GGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPPADALKLRSPRGSPDGSLQTGKPSAPKKLKLDVGEAMAPPSHRKGDSNANSDVCAAALRGLSRTILMCGPLIKEETHRRLHDLVLPLVMGVQQGEVLGSSPYTSSRCRRELYCLLLALLLAPSPRC

SEQ ID NO: 32 - protein, 495 aa, Homo sapiens
GGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPPADALKLRSPRGSPDGSLQTGKPSAPKKLKLDVGEAMAPPSHRKGDSNANSDVCAAALRGLSRTILMCGPLIKEETHRRLHDLVLPLVMGVQQGEVLGSSPYTSSR

SEQ ID NO: 33 - protein, 12 aa, Homo sapiens
SHRKGDSNANSD